(19) United States
(12) Patent Application Publication
(10) Pub. No.: US 2024/0352530 A1
(43) Pub. Date: Oct. 24, 2024

(54) CIRCULATING MICRORNA PANEL FOR THE EARLY DETECTION OF BREAST CANCER AND METHODS THEREOF

(71) Applicants: National University of Singapore, Singapore (SG); Singapore Health Services Pte. Ltd., Singapore (SG); MiRXES Lab Pte. Ltd., Singapore (SG); National University Hospital (Singapore) Pte Ltd, Singapore (SG)

(72) Inventors: Mikael Hartman BO ANDERS, Singapore (SG); Heng-Phon TOO, Singapore (SG); Siew Gek Ann LEE-LIM, Singapore (SG); Ruiyang ZOU, Singapore (SG); Lihan ZHOU, Singapore (SG); He CHENG, Singapore (SG)

(73) Assignees: National University of Singapore, Singapore (SG); Singapore Health Services Pte. Ltd., Singapore (SG); MiRXES Lab Pte. Ltd., Singapore (SG); National University Hospital (Singapore) Pte. Ltd., Singapore (SG)

(21) Appl. No.: 18/293,816

(22) PCT Filed: Aug. 2, 2022
(86) PCT No.: PCT/SG2022/050552

§ 371 Date: Jan. 31, 2024

(30) Foreign Application Priority Data
Aug. 2, 2021 (SG) 10202108448V
Aug. 2, 2022 (WO) PCT/SG2022/050552

(51) Int. Cl. C12Q 1/6886

(52) U.S. Cl. CPC C12Q 1/6886; C12Q 2600/118; C12Q 2600/178; G01N 2800/50

(57) ABSTRACT

Disclosed herein is a method for determining whether a subject is suffering from, or is at risk of developing breast cancer, wherein the method comprises detecting differential expression levels of at least two or more miRNA markers from a biological sample. Also disclosed herein is a method of determining whether a subject is suffering from, or is at risk of developing, breast cancer based on miRNA expression.

### FIELD OF THE INVENTION

The present invention relates generally to the field of molecular biology. In particular, the present invention relates to the use of biomarkers for the detection and diagnosis of cancer.

### BACKGROUND OF THE DISCLOSURE

Mammography has been widely used as a screening tool for breast cancer, despite its high false-positive rate and its lack of sensitivity in detecting cancer in dense breasts. A high rate of false positivity of 11 to 12% has been detected among women in the United States who have undergone mammographic screening. MiRNAs are deemed suitable as biomarkers because of altered miRNA expression profiles in cancer that reflect disease development, as well as the stability and the accessibility of circulating miRNAs in a myriad of body fluids including blood, urine and saliva. Minimally invasive methods, such as miRNA-based liquid biopsies, can potentially overcome these disadvantages and improve overall detection accuracy.

Thus, there is an unmet need for a minimally invasive method of detecting and predicting the onset of breast cancer in a subject.

### SUMMARY

In one aspect, the present disclosure refers to a method for determining whether a subject is suffering from, or is at risk of developing breast cancer. The method comprises detecting differential expression levels of at least two or more miRNA markers from a biological sample obtained from the subject. The miRNAs are selected from miR-133a-3p, miR-497-5p, miR-24-3p, miR-125b-5p, miR-377-3p, miR-374c-5p, miR-324-5p and miR-19b-3p, and said differential expression level is compared with that of a cancer-free subject.

In another aspect, the present disclosure refers to a method of treating breast cancer. The method comprises i) detecting the presence of miRNA in a bodily fluid sample obtained from the subject; ii) measuring the expression level of at least two miRNA in the bodily fluid sample; and iii) using a prediction algorithm score based on the differential expression level of the miRNAs measured previously to predict the probability of the subject to suffer from or develop breast cancer. The at least two or more miRNA markers are selected from miR-133a-3p, miR-497-5p, miR-24-3p, miR-125b-5p, miR-377-3p, miR-374c-5p, miR-324-5p and miR-19b-3p, and the differential expression of miR-377-3p, miR-374c-5p, miR-324-5p and miR-19b-3p, if present, are downregulated, as compared to a control, or wherein the differential expression of miR-133a-3p, miR-497-5p, mir-24-3p, and miR-125b-5p, if present, are upregulated, as compared to a control. The method further comprises determining the subject to suffer from breast cancer or to be at risk of developing breast cancer, and treating the subject determined to suffer from breast cancer or determined to be at risk of developing breast cancer with an anti-breast cancer compound. In this method, the control for comparing the expression level of the at least two miRNAs referred to in step ii) is a breast cancer-free subject.

In yet another aspect, the present disclosure refers to a kit for use in the method as described herein.

### BRIEF DESCRIPTION OF THE DRAWINGS

The invention will be better understood with reference to the detailed description when considered in conjunction with the non-limiting examples and the accompanying drawings, in which:

FIG. 1 provides an overview of the biomarker discovery based on large comprehensive datasets comprising Caucasian and Asian patient groups. FIG. 1A is a volcano plot for 324 miRNAs profiled in 183 breast cancer patients as compared to 106 healthy individuals in the discovery cohort. Eighty-six miRNAs with p-values of less than 0.01 and magnitudes of log 2 (fold change) of more than 0.5 are highlighted. FIG. 1B is a heat-map of 663 cancer and non-cancer samples clustered using the expression of 33 selected miRNA biomarkers identified in the Discovery cohort. The expression levels (copy/ml) of miRNAs are presented in log 2 scale and standardized to zero mean. The grayscale represents the concentrations of miRNA. Hierarchical clustering was carried out for both dimensions (miRNAs: Y-axis, samples: X-axis) based on the Euclidean distance. Correlation of log 2 (fold change) for 33 selected miRNA biomarker candidates in the Discovery cohort and Validation 1 cohort is shown in FIG. 1C. The miRNA biomarker candidates with p-values larger than 0.05 in the validation 1 cohort are highlighted. This figure summarized the process of discovery and validation of 33 circulating serum miRNAs associated with breast cancer that were identified from the patient samples.

FIG. 2 summarizes further processes of obtaining multi-miRNA biomarker panels based on the patient data. FIG. 2A shows boxplots of AUC (area under the curve) values obtained for multi-miRNA biomarker panels (with 2-12 miRNAs), in both the training and test sets, calculated from 200 iterations of the two-fold cross-validation process. The boxplot presents the 25th, 50th, and 75th percentiles in panel AUC. In the columns of FIG. 2B, the median AUC for the training and test sets from 200 iterations of the two-fold cross-validation process for multi-miRNA panels with 2-12 miRNAs are presented. *** p<0.001 (Student's t-test). FIG. 2C demonstrates the ROC (receiver operating characteristic) curves for breast cancer prediction performance of the optimal eight-miRNA biomarker panel in the Discovery and Validation 1 cohorts. The point with the maximum classification accuracy is shown as the box on the curve. The sensitivity and specificity values at the maximum accuracy point are also shown. The 95% CI (confidence interval) for these values is shown in the brackets. Through optimization steps, the performance of the multi-miRNA biomarker panels from the combinations of at least two miRNA biomarkers to 12 biomarkers is further evaluated.

FIG. 3 shows results of the validation of an eight-miRNA biomarker panel, amongst other multi-miRNA panels. FIG. 3A shows the ROC curve for breast cancer prediction performance of the optimal eight-miRNA biomarker panel in the Validation 2 cohort. The point with the maximum classification accuracy is shown as the box on the curve. The sensitivity and specificity values at the maximum accuracy point are also shown. The 95% CI (confidence interval) for these values is shown in the brackets. FIG. 3B shows a column graph of the AUC value of the eight-miRNA biomarker panel in detecting breast cancer in the Validation 1 and Validation 2 cohorts, separated by sample source. FIG. 3C shows the ROC curves for performance of the optimal eight-miRNA biomarker panel in predicting early (stages 0, I and II) and late (stages III and IV) breast cancer in the Validation 2 cohort. The data shown in FIG. 3 demonstrates a high sensitivity and specificity for the eight-miRNA panel, and comparable sensitivity for different ethnicity of patients. FIG. 3 also shows that the eight-miRNA panel can distinguish different stages of cancer development from cancer-free individuals.

FIG. 4 summarizes the calculation of prediction algorithm scores based on expression of the miRNA comprised in the eight-miRNA biomarker panel. FIG. 4A shows boxplots depicting prediction algorithm scores of cancer and non-cancer samples, calculated based on expression levels of the miRNA present in the eight-miRNA panel. FIG. 4B shows boxplots depicting the prediction algorithm scores of non-cancer samples and cancer samples by tumour stage (0, I, II, III, IV, and unknown). This data shows that the prediction algorithm score serves as an indicator to predict whether an individual is suffering or is at risk of developing cancer based on an eight-miRNA biomarker panel.

FIG. 5 shows heatmaps depicting the relative expression levels of 324 candidate miRNAs in serum of both breast cancer cases and non-cancer controls. The geNORM (geNORM, RRID:SCR_006763) 22 and NormFinder (NormFinder, RRID:SCR_003387) 23 software were used to identify endogenous reference miRNAs showed stable expression across all samples and could be used to normalize for varying sample RNA inputs for RT-qPCR. Three miRNAs with stable expression were identified and used to normalize the expression levels of miRNAs across samples: miR-128-3p, miR-652-3p, and miR-106b-3p. The numbers on the x-axis for both subfigures are the numbers 50, 100, 150, 200, and 250, respectively.

FIG. 6 is a schematic summarising the workflow disclosed herein, which comprises one discovery cohort and two validation cohorts. Serum samples from six different centres across Europe, USA and Singapore were collected, processed, and analysed in three cohorts. FIG. 6 demonstrates how two-step validation is used in developing and obtaining an eight-miRNA biomarker panel of high specificity and sensitivity.

### DEFINITIONS

As used herein, the term “miRNA” refers to microRNA, small non-coding RNA molecules, which in some examples contain about 19 to 25 nucleotides, and are found in plants, animals and some viruses. miRNAs are known to have functions in RNA silencing and post-transcriptional regulation of gene expression. These highly conserved RNAs regulate the expression of genes by binding to the 3′-untranslated regions (3′-UTR) of specific mRNAs. For example, each miRNA is thought to regulate multiple genes, and since hundreds of miRNA genes are predicted to be present in higher eukaryotes. miRNAs tend to be transcribed from several different loci in the genome. These genes encode for long RNAs with a hairpin structure that when processed by a series of RNaseIII enzymes (including Drosha and Dicer) form a miRNA duplex of usually about 19 to 25 nucleotides long with 2 nucleotide overhangs on the 3′end.

As used herein, the term “differential expression” refers to the measurement of a cellular component in comparison to a control or another sample, and thereby determining the difference in, for example concentration, presence or intensity of said cellular component. The result of such a comparison can be given in the absolute, that is a component is present in the samples and not in the control, or in the relative, that is the expression or concentration of component is increased or decreased compared to the control. The terms “increased” and “decreased” in this case can be interchanged with the terms “upregulated” and “downregulated” which are also used in the present disclosure. In the context of the present disclosure, differential expression in conjunction with expression levels refers to the concentration of products of gene expression of a particular gene. Such products of gene expression can be, but are not limited to, for example, RNA, mRNA, and/or protein.

As used herein, the term “HER” or “HER2” refers to the human epidermal growth factor 2, a member of the human epidermal growth factor receptor (HER/EGFR/ERBB) family involved in normal cell growth. It is found on some types of cancer cells, including, but not limited to, breast and ovarian cancer cells. Cancer cells removed from the body may be tested for the presence of HER2/neu to help identify an effective treatment modality. HER2 is also often referred to as receptor tyrosine-protein kinase erbB-2, CD340, and human epidermal growth factor receptor 2.

As used herein, the term “Luminal A” or “LA” refers to a sub-classification of breast cancers according to a multitude of genetic markers. A breast cancer can be determined to be luminal A or luminal B, in addition to being estrogen receptor (ER) positive, progesterone receptor (PR) positive and/or hormone receptor (HR) negative, among others. Clinical definition of a luminal A cancer is a cancer that is ER positive and PR positive, but negative for HER2. Luminal A breast cancers are likely to benefit from hormone therapy and may also benefit from chemotherapy. A luminal B cancer is a cancer that is ER positive, PR negative and HER2 positive. Luminal B breast cancers are likely to benefit from chemotherapy and may benefit from hormone therapy and treatment targeted to HER2.

As used herein, the term “triple negative” or “TN” refers to a breast cancer, which had been tested and found to lack (or be negative) for hormone epidermal growth factor receptor 2 (HER-2), estrogen receptors (ER), and progesterone receptors (PR). Since triple negative tumour cancers lack the necessary receptors, common treatments, for example hormone therapy and drugs that target estrogen, progesterone, and HER-2, are ineffective. Using chemotherapy to treat triple negative breast cancer is still an effective option. In fact, a triple negative breast cancer may respond even better to chemotherapy in the earlier stages than many other forms of cancer.

As used herein, where a subject is diagnosed with breast cancer or the onset of breast cancer, the term “treatment” to breast cancer may include, but is not limited to: surgery, radiation therapy, chemotherapy, hormone therapy (e.g. tamoxifen, luteinizing hormone-releasing hormone (LHRH) agonist or an aromatase inhibitor), targeted therapy (such as monoclonal antibodies (e.g. trastuzumab, pertuzumab or sacituzumab govitecan), tyrosine kinase inhibitor (e.g. tucatinib, neratinib or laptinib), cycline-dependent kinase inhibitors (e.g. palboiclib, ribociclib etc), mTOR inbitors (e.g. everolimus), PARP inhibitors (e.g. Olaparib, Talazoparib, etc) immunotherapy (e.g. PD-1 and PDL-1 inhibitors) or any anti-breast cancer compounds. It is also known in the art that early detection of cancer significantly improves the survival rate compared to subjects where the cancer is detected in the late stage, hence highlighting the importance of early detection. Hence, an effective test, such as a liquid biopsy test, able to detect breast cancer with high specificity and sensitivity would greatly assist early detection of the disease or the onset of the disease.

As used herein, the term “(statistical) classification” refers to the problem of identifying to which of a set of categories (sub-populations) a new observation belongs, on the basis of a training set of data containing observations (or instances) whose category membership is known. An example is assigning a diagnosis to a given patient as described by observed characteristics of the patient (gender, blood pressure, presence or absence of certain symptoms, etc.). In the terminology of machine learning, classification is considered an instance of supervised learning, i.e. learning where a training set of correctly identified observations is available. The corresponding unsupervised procedure is known as clustering, and involves grouping data into categories based on some measure of inherent similarity or distance. Often, the individual observations are analysed into a set of quantifiable properties, known variously as explanatory variables or features. These properties may variously be categorical (e.g. “A”, “B”, “AB” or “O”, for blood type), ordinal (e.g. “large”, “medium” or “small”), integer-valued (e.g. the number of occurrences of a part word in an email) or real-valued (e.g. a measurement of blood pressure). Other classifiers work by comparing observations to previous observations by means of a similarity or distance function. An algorithm that implements classification, especially in a concrete implementation, is known as a classifier. The term “classifier” sometimes also refers to the mathematical function, implemented by a classification algorithm, which maps input data to a category.

As used herein, the term “pre-trained” or “supervised (machine) learning” refers to a machine learning task of inferring a function from labelled training data. The training data can consist of a set of training examples. In supervised learning, each example is a pair consisting of an input object (typically a vector) and a desired output value (also called the supervisory signal). A supervised learning algorithm, that is the algorithm to be trained, analyses the training data and produces an inferred function, which can be used for mapping new examples. An optimal scenario will allow for the algorithm to correctly determine the class labels for unseen instances. This requires the learning algorithm to generalize from the training data to unseen situations in a “reasonable” way.

As used herein, the term “score” refers to an integer or number, that can be determined mathematically, for example by using computational models a known in the art, which can include but are not limited to, SMV, as an example, and that is calculated using any one of a multitude of mathematical equations and/or algorithms known in the art for the purpose of statistical classification. Such a score is used to enumerate one outcome on a spectrum of possible outcomes. The relevance and statistical significance of such a score depends on the size and the quality of the underlying data set used to establish the results spectrum. For example, a blind sample may be input into an algorithm, which in turn calculates a score based on the information provided by the analysis of the blind sample. This results in the generation of a score for said blind sample. Based on this score, a decision can be made, for example, how likely the patient, from which the blind sample was obtained, has cancer or not. The ends of the spectrum may be defined logically based on the data provided, or arbitrarily according to the requirement of the experimenter. In both cases the spectrum needs to be defined before a blind sample is tested. As a result, the score generated by such a blind sample, for example the number “45” may indicate that the corresponding patient has cancer, based on a spectrum defined as a scale from I to 50, with “1” being defined as being cancer-free and “50” being defined as having cancer.

A description of breast cancer stages as described by the National Cancer Institute at the national Institutes of Health are as follows:

Stage 0 (carcinoma in situ): there are 3 types of breast carcinoma in situ: Ductal carcinoma in situ (DCIS) is a non-invasive condition in which abnormal cells are found in the lining of a breast duct. The abnormal cells have not spread outside the duct to other tissues in the breast. In some cases, DCIS may become invasive cancer and spread to other tissues. At this time, there is no way to know which lesions could become invasive. Lobular carcinoma in situ (LCIS) is a condition in which abnormal cells are found in the lobules of the breast. This condition seldom becomes invasive cancer. Paget disease of the nipple is a condition in which abnormal cells are found in the nipple only.

Stage 1: In stage I, cancer has formed. Stage I is divided into stages IA and IB. In stage IA, the tumour is 2 centimetres or smaller. Cancer has not spread outside the breast. In stage IB, small clusters of breast cancer cells (larger than 0.2 millimetres but not larger than 2 millimetres) are found in the lymph nodes and either: no tumour is found in the breast; or the tumour is 2 centimetres or smaller.

Stage II: Stage II is divided into stages IIA and IIB. In stage IIA: no tumour is found in the breast or the tumour is 2 centimetres or smaller. Cancer (larger than 2 millimetres) is found in 1 to 3 axillary lymph nodes or in the lymph nodes near the breastbone (found during a sentinel lymph node biopsy); or the tumour is larger than 2 centimetres but not larger than 5 centimetres.

Cancer has not spread to the lymph nodes. In stage IIB, the tumour is: larger than 2 centimetres but not larger than 5 centimetres. Small clusters of breast cancer cells (larger than 0.2 millimetres but not larger than 2 millimetres) are found in the lymph nodes; or larger than 2 centimetres but not larger than 5 centimetres. Cancer has spread to 1 to 3 axillary lymph nodes or to the lymph nodes near the breastbone (found during a sentinel lymph node biopsy); or larger than 5 centimetres. Cancer has not spread to the lymph nodes.

Stage III: Stage III is divided into stages IIIA, IIIB and IIIC. In stage IIIA: no tumour is found in the breast or the tumour may be any size. Cancer is found in 4 to 9 axillary lymph nodes or in the lymph nodes near the breastbone (found during imaging tests or a physical exam); or the tumour is larger than 5 centimetres. Small clusters of breast cancer cells (larger than 0.2 millimetres but not larger than 2 millimetres) are found in the lymph nodes; or the tumour is larger than 5 centimetres. Cancer has spread to 1 to 3 axillary lymph nodes or to the lymph nodes near the breastbone (found during a sentinel lymph node biopsy). In stage IIIB: the tumour may be any size and cancer has spread to the chest wall and/or to the skin of the breast and caused swelling or an ulcer. Also, cancer may have spread to: up to 9 axillary lymph nodes; or the lymph nodes near the breastbone. Cancer that has spread to the skin of the breast may also be inflammatory breast cancer. In stage IIIC: no tumour is found in the breast or the tumour may be any size. Cancer may have spread to the skin of the breast and caused swelling or an ulcer and/or has spread to the chest wall. Also, cancer has spread to: 10 or more axillary lymph nodes; or lymph nodes above or below the collarbone; or axillary lymph nodes and lymph nodes near the breastbone.

Stage IV: In stage IV, cancer has spread to other organs of the body, most often the bones, lungs, liver, or brain.

Generally speaking, the term “early stage” cancer is used herein to refer to cancer of stage 0, I, or II. The term “late-stage” is used to describe a cancer of the stage III or IV.

### DETAILED DESCRIPTION OF DISCLOSURE

MiRNAs are evolutionary conserved, single-stranded non-coding RNAs of 19 to 25 nucleotides which primarily function in mediating the degradation or translational repression of mRNA targets. Under normal physiological conditions, miRNAs are key components of feedback mechanisms for a wide range of biological pathways such as cell proliferation, differentiation and apoptosis. Conversely, dysregulated miRNAs have been implicated in the hallmarks of cancer including supporting tumour growth by inhibiting growth suppression, sustaining proliferative signalling and resisting cell death, activating invasion and metastasis, and promoting angiogenesis. It is now known that miRNAs regulate oncogenesis through their tumour suppressor or oncogenic activities, with increasing evidence of aberrant miRNA expression in a variety of malignancies.

In order to improve breast cancer detection, numerous blood-derived miRNA biomarkers with increased discriminative ability as compared to mammography, have been reported in recent years. The miRNAs miR-145, miR-21 and miR-221 are among the more frequently reported candidates and demonstrate potential for the early detection of breast cancer.

However, to date, none of the previously published miRNA biomarker studies for breast cancer have proceeded to biomarker clinical trials due to various shortcomings. For example, the majority of previously published circulating miRNA biomarker studies for breast cancer were conducted in smaller sample sizes comprising of a single ethnic group, and with one or no validation phase.

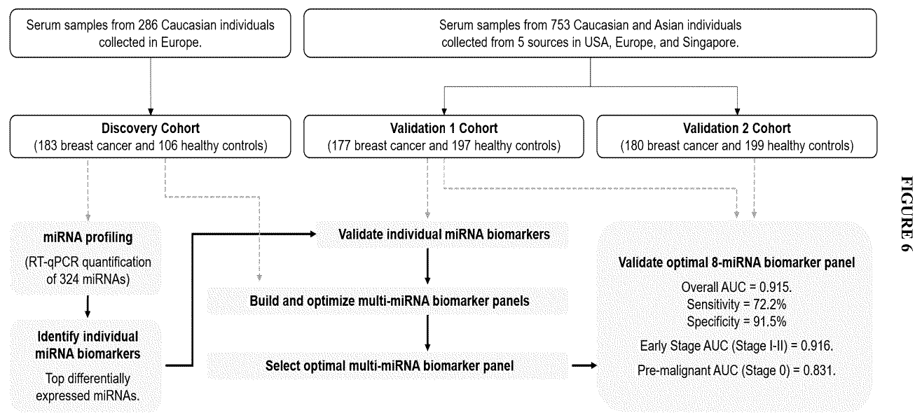

In the present disclosure, a multi-centre case-control study is discussed, which had been carried out in three phases: one discovery phase (n=289) and two validation phases (n=374 and n=379) (FIG. 6). The three patient cohorts used (Discovery Cohort, Validation 1 Cohort, and Validation 2 Cohort, respectively) comprised samples from six different sources (Table 1). The Discovery cohort comprised European Caucasian samples obtained from the Asterand biobank (designated as Source 1) while the Validation 1 and 2 cohorts were a mix of Caucasian and Asian samples from five different sources in USA, Ukraine, Russia, and Singapore (designated as Sources 2-5) (Table 1). The Discovery and Validation cohorts included female subjects aged 40 to 75 years diagnosed with stage 1 to stage 3 breast cancer of all subtypes (Table 2). The inclusion criteria for cases were women diagnosed with breast cancer, and the inclusion criteria for controls was no history of cancer in healthy female individuals. A blinded approach was not done as the study design included two validation phases. Written informed consent was obtained from all participants and the research was approved by all relevant Institutional Review Boards (IRBs). Samples from the Asterand and Tissue Solution biobanks were ethically collected under IRB-approved protocols and fully consented.

TABLE 1
Patient cohorts used in study

| | | | Cohort: | | | | | |
|---|---|---|---|---|---|---|---|---|
| | | | Discovery | | Validation 1 | | Validation 2 | |
| | | | Non-cancer (NC)/Cancer (C): | | | | | |
| | | | NC | C | NC | C | NC | C |
| Source | | | 106 | 183 | 197 | 177 | 199 | 180 |
| | | Ethnicity | | | | | | |
| 1 | Asterand | Caucasian | 106 | 183 | — | — | — | — |
| | biobank | | (100%) | (100%) | | | | |
| | (Europe) | | | | | | | |
| 2 | Asterand | Caucasian | — | — | 39 | 39 | 39 | 40 |
| | biobank | | | | (19%) | (22%) | (19%) | (22%) |
| | (USA) | | | | | | | |
| 3 | Tissue | Caucasian | — | — | 33 | 23 | 34 | 24 |
| | Solutions | | | | (17%) | (13%) | (17%) | (13%) |
| | biobank | | | | | | | |
| | (Ukraine) | | | | | | | |
| 4 | Tissue | Caucasian | — | — | 47 | 48 | 47 | 48 |
| | Solutions | | | | (24%) | (27%) | (24%) | (27%) |
| | biobank | | | | | | | |
| | (Russia) | | | | | | | |
| 5 | National | Asian | — | — | 35 | 38 | 35 | 38 |
| | University | (Singapore) | | | (18%) | (21%) | (18%) | (21%) |
| | Hospital | | | | | | | |
| 6 | National | Asian | — | — | 43 | 29 | 44 | 30 |
| | Cancer | (Singapore) | | | (22%) | (16%) | (22%) | (17%) |
| | Centre | | | | | | | |
| | Singapore | | | | | | | |
| | | Age (years) | | | | | | |
| | | Mean | 53.7 | 52.4 | 53.0 | 56.6 | 55.5 | 55.4 |
| | | Median | 53 | 51 | 52 | 57 | 56 | 55 |
| | | Range | 42-65 | 30-85 | 29-82 | 31-77 | 26-83 | 28-87 |
| | | Sex | | | | | | |
| | | Male | 0 | 0 | 0 | 0 | 0 | 0 |
| | | Female | 106 | 183 | 197 | 177 | 199 | 180 |
| | | Cancer Stage | | | | | | |
| | | 0 | — | 0 | — | 16 | — | 23 |
| | | | | (0%) | | (9%) | | (13%) |
| | | I | — | 77 | — | 51 | — | 45 |
| | | | | (42%) | | (29%) | | (25%) |
| | | II | — | 78 | — | 61 | — | 58 |
| | | | | (43%) | | (34%) | | (32%) |
| | | III | — | 28 | — | 5 | — | 8 |
| | | | | (15%) | | (3%) | | (4%) |
| | | IV | — | 0 | — | 3 | — | 3 |
| | | | | (0%) | | (2%) | | (2%) |
| | | Unknown | — | 0 | — | 41 | — | 43 |
| | | | | (0%) | | (23%) | | (24%) |

Quantitative RT PCR profiling of 324 miRNAs was performed on serum samples from breast cancer (all stages) and healthy subjects to identify miRNA biomarkers. Two-fold cross-validation was used for building and optimizing breast cancer-associated miRNA biomarker panels. A panel was validated in cohorts with Caucasian and Asian samples. Diagnostic ability was evaluated using area under the curve (AUC) analysis.

Thirty (30) upregulated or downregulated miRNAs had been identified and validated in breast cancer.

An eight-miRNA biomarker panel showed consistent performance in all cohorts and was validated with AUC, accuracy, sensitivity, and specificity of 0.915, 82.3%, 72.2% and 91.5%, respectively. The prediction model detected breast cancer in both Caucasian and Asian populations with AUCs ranging from 0.880-0.973, including pre-malignant lesions (stage 0; AUC of 0.831) and early-stage (stages I-II) cancers (AUC of 0.916).

Based on the data disclosed herein, a prediction model for breast cancer, applicable for Caucasian and Asian populations and patients of various cancer stages, was established. The miRNA-based prediction model disclosed herein represents an alternative modality for breast cancer screening, thereby reducing the number of biopsies resulting from false-positive mammograms.

Thus, the method disclosed herein can be used in conjunction or together with methods known in the art for identifying the presence of breast cancer. In one example, the method disclosed herein is used in combination with other breast cancer screening or diagnostic methods, such as, but not limited to mammography, ultrasound, magnetic resonance imaging, and combinations thereof. In another example, the method disclosed herein, whether used alone or in combination with other breast cancer screening or diagnostic methods, identifies subjects at risk of suffering from breast cancer that would be further subjected to a biopsy.

Also disclosed herein is a kit for use according to the methods described herein. Such a kit can be used with methods such as, but not limited to, a quantitative reverse-transcription real-time polymerase chain reaction (qRT-PCR), a locked nucleic acid (LNA) real-time PCR, sequencing, a northern blotting, a hybridization, a CRISPR gene editing, a micro-array assay, and combinations thereof.

Thus, in one example, there is disclosed a method for determining whether a subject is suffering from or is at risk of developing breast cancer. In one example, the method comprises detecting differential expression levels of at least two or more miRNA markers from a biological sample obtained from the subject. In another example, the differential expression level is compared with that of a cancer-free subject.

Samples used herein were obtained from subjects and comprise, for example, bodily fluids as well as solid components. Thus, in one example, the method disclosed herein is performed on a biological sample. In another example, the method disclosed herein is performed on a biological sample obtained from a subject. In yet another example, the biological sample is a bodily fluid.

Examples of bodily fluids are, but are not limited to, cellular and/or non-cellular components of a liquid biopsy, amniotic fluid, a bronchial lavage, cerebrospinal fluid, interstitial fluid, peritoneal fluid, pleural fluid, saliva, seminal fluid, urine, a tear, peripheral blood, whole blood, plasma, and serum. In one example, the bodily fluid is plasma. In another example, the bodily fluid is serum.

TABLE 2
Additional information for cancer patient cohorts

| Cohort: | Discovery | | Validation 1 | | Validation 2 | |
|---|---|---|---|---|---|---|
| Cancer Samples: | 183 | | 177 | | 180 | |
| Estrogen Receptor | | | | | | |
| (ER) Status | | | | | | |
| ER Positive | 90 | (49.2%) | 78 | (44.1%) | 95 | (52.8%) |
| ER Negative | 93 | (50.8%) | 37 | (20.9%) | 18 | (10.0%) |
| Unknown | 0 | (0%) | 62 | (35.0%) | 67 | (37.2%) |
| Progesterone Receptor | | | | | | |
| (PR) Status | | | | | | |
| PR Positive | 90 | (49.2%) | 67 | (37.9%) | 77 | (42.8%) |
| PR Negative | 93 | (50.8%) | 48 | (27.1%) | 36 | (20.0%) |
| Unknown | 0 | (0%) | 62 | (35.0%) | 67 | (37.2%) |
| HER2 Status | | | | | | |
| HER2 Positive | 46 | (25.1%) | 29 | (16.4%) | 18 | (10.0%) |
| HER2 Negative | 137 | (74.9%) | 53 | (29.9%) | 62 | (34.4%) |
| HER2 Equivocal | 0 | (0%) | 13 | (7.3%) | 12 | (6.7%) |
| Unknown | 0 | (0%) | 62 | (35.0%) | 67 | (37.2%) |
| Subtype | | | | | | |
| Luminal A | 90 | (49.2%) | 47 | (26.6%) | 57 | (31.7%) |
| Luminal B | 0 | (0%) | 10 | (5.6%) | 10 | (5.6%) |
| Triple Negative (TNBC) | 47 | (25.7%) | 6 | (3.4%) | 5 | (2.8%) |
| HER2-enriched | 46 | (25.1%) | 19 | (10.7%) | 8 | (4.4%) |
| Unknown | 0 | (0%) | 95 | (53.7%) | 100 | (55.6%) |

### Biomarker Identification

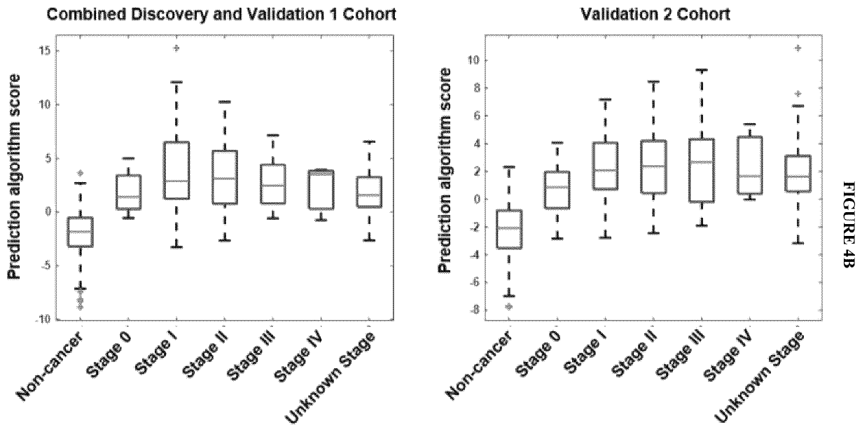

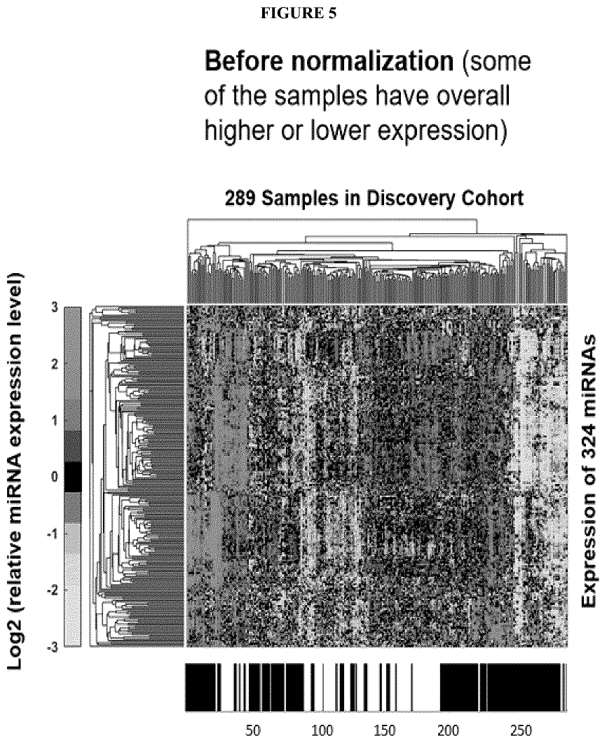

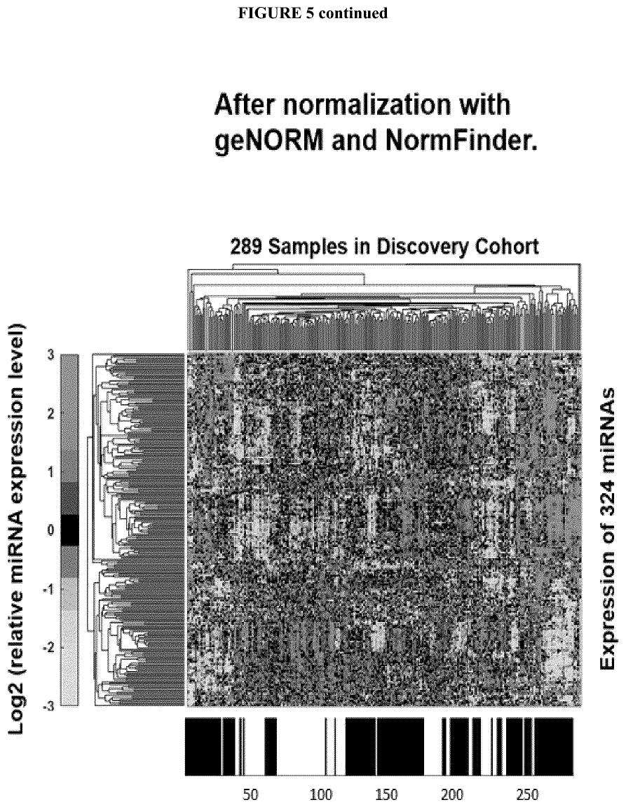

The absolute quantities of 324 candidate miRNAs in the serum of both breast cancer cases and non-cancer controls were determined. The geNORM (geNORiM, RRID:SCR_006763) and NormFinder (NormFinder, RRID:SCR_003387) software were used to identify endogenous reference miRNAs that had stable expression across all samples and could be used to normalize for varying sample RNA inputs for RT-qPCR. Three miRNAs with stable expression were identified and used to normalize the expression levels of miRNAs across samples: miR-128-3p, miR-652-3p, and miR-106b-3p (FIG. 5). The normalized miRNA expression values were used to compare the expression levels of individual miRNAs between breast cancer cases and non-cancer controls. Unsupervised hierarchical clustering was carried out based on Euclidean distance of normalized miRNA expression levels in two dimensions (samples and miRNA expression). The top miRNAs with p<0.01 and magnitude of log 2 fold change >0.5 were selected for validation using the Validation 1 cohort. Statistical significance of differences in miRNA expression was determined using Student's t-test. All p-values were corrected for multiple hypotheses testing using false discovery rate (FDR) adjustment. Those miRNAs which were differentially expressed in both the Discovery and Validation 1 cohorts were considered validated. A relaxed cut-off of p<0.05 with magnitude of log 2 fold change >0.5 was used to identify validated miRNAs for biomarker panel building and optimization (Table 3).

TABLE 3
Relative expression levels and individual performance of
the miRNAs included in an exemplary eight-miRNA panel

| | | log2(Fold-Change) | |
|---|---|---|---|
| | p-value (cancer vs | (cancer vs non- | |
| miRNA | non-cancer) | cancer) | AUC |
| biomarker | Discovery Cohort | Discovery Cohort | Discovery Cohort |
| miR-377-3p | 1.01E−09 | 0.5684 | 0.7082 |
| miR-374c- | 2.39E−13 | −1.1143 | 0.7658 |
| 5p | | | |
| miR-324-5p | 1.97E−17 | −0.7877 | 0.8186 |
| miR-24-3p | 4.70E−25 | 0.9359 | 0.8478 |
| miR-133a- | 8.32E−08 | 1.3679 | 0.6513 |
| 3p | | | |
| miR-125b- | 1.48E−14 | 1.0654 | 0.767 |
| 5p | | | |
| miR-497-5p | 1.74E−15 | 0.7419 | 0.7972 |
| miR-19b-3p | 2.17E−12 | 0.6982 | 0.7465 |

| | | log2(Fold-Change) | |
|---|---|---|---|
| | p-value (cancer vs | (cancer vs non- | |
| miRNA | non-cancer) | cancer) | AUC |
| biomarker | Validation 1 Cohort | Validation 1 Cohort | Validation 1 Cohort |
| miR-377-3p | 8.09E−03 | 0.1886 | 0.5927 |
| miR-374c- | 1.29E−11 | −0.9278 | 0.7009 |
| 5p | | | |
| miR-324-5p | 9.55E−12 | −0.6003 | 0.6908 |
| miR-24-3p | 1.42E−13 | 0.5066 | 0.7175 |
| miR-133a- | 6.42E−04 | 0.5592 | 0.5665 |
| 3p | | | |
| miR-125b- | 3.91E−04 | 0.4145 | 0.6098 |
| 5p | | | |
| miR-497-5p | 1.87E−03 | 0.1684 | 0.5874 |
| miR-19b-3p | 5.85E−05 | 0.246 | 0.6195 |
| | | log2(Fold-Change) | |
| | p-value (cancer vs | (cancer vs non- | |
| miRNA | non-cancer) | cancer) | AUC |
| biomarker | Validation 2 Cohort | Validation 2 Cohort | Validation 2 Cohort |
| miR-377-3p | 3.45E−02 | 0.1466 | 0.5558 |
| miR-374c- | 8.97E−13 | −0.9892 | 0.7047 |
| 5p | | | |
| miR-324-5p | 2.88E−14 | −0.6709 | 0.7262 |
| miR-24-3p | 1.49E−20 | 0.6196 | 0.7591 |
| miR-133a- | 5.14E−02 | 0.3056 | 0.5224 |
| 3p | | | |
| miR-125b- | 2.59E−04 | 0.4079 | 0.6245 |
| 5p | | | |
| miR-497-5p | 2.08E−02 | 0.1319 | 0.5809 |
| miR-19b-3p | 1.22E−05 | 0.2584 | 0.6303 |

Although an AUC value of 0.971 has been reported by a five-miRNA signature (miR-1246, miR-1307-3p, miR-4634, miR-6861-5p and miR-6875-5p) panel reported previously, it is noted that the five-miRNA panel previously disclosed was based primarily on microarray profiling, a method which is known to have poor specificity, and that only one miRNA, miR-1246, was validated by qRT-PCR using 26 serum samples. Instead, the panel of eight miRNA markers disclosed herein are all validated by qPCR, which has a higher specificity.

In another example, the at least 8 miRNA markers are selected from miR-133a-3p, miR-497-5p, miR-24-3p, miR-125b-5p, miR-377-3p, miR-374c-5p, miR-324-5p and miR-19b-3p.

In one example, the miRNA panels disclosed herein comprise groups of 4 miRNA, wherein the miRNA are, but are not limited to, miR-133a-3p, miR-497-5p, miR-24-3p, miR-125b-5p, miR-377-3p, miR-374c-5p, miR-324-5p and miR-19b-3p.

Table 4 below provides the mean (a) and median (b) AUCs of multivariate panels comprising combinations of 2 to 8 miRNA where one of the miRNAs were fixed and combined with 1 to 7 additional miRNAs selected from miR-133a-3p, miR-497-5p, miR-24-3p, miR-125b-5p, miR-377-3p, miR-374c-5p, miR-324-5p and miR-19b-3p.

TABLE 4

| | Fixed | mean AUC of panel with the selected miRNA | | | | | | |
|---|---|---|---|---|---|---|---|---|
| | miRNA | 2 | 3 | 4 | 5 | 6 | 7 | 8 |
| No | biomarker | miRNA | miRNA | miRNA | miRNA | miRNA | miRNA | miRNA |

(a)

| | | | | | | | | |
|---|---|---|---|---|---|---|---|---|
| 1 | miR-377-3p | 0.667 | 0.733 | 0.786 | 0.830 | 0.868 | 0.868 | 0.899 |
| 2 | miR-374c-5p | 0.794 | 0.825 | 0.849 | 0.870 | 0.889 | 0.889 | 0.908 |
| 3 | miR-324-5p | 0.758 | 0.797 | 0.828 | 0.855 | 0.880 | 0.880 | 0.903 |
| 4 | miR-24-3p | 0.730 | 0.775 | 0.814 | 0.847 | 0.876 | 0.876 | 0.902 |
| 5 | miR-133a-3p | 0.677 | 0.742 | 0.794 | 0.835 | 0.870 | 0.870 | 0.900 |
| 6 | miR-125b-5p | 0.699 | 0.748 | 0.794 | 0.835 | 0.871 | 0.871 | 0.900 |
| 7 | miR-497-5p | 0.674 | 0.735 | 0.787 | 0.830 | 0.866 | 0.866 | 0.898 |
| 8 | miR-19b-3p | 0.678 | 0.739 | 0.789 | 0.832 | 0.868 | 0.868 | 0.899 |

(b)

| | | | | | | | | |
|---|---|---|---|---|---|---|---|---|
| 1 | miR-377-3p | 0.658 | 0.746 | 0.796 | 0.839 | 0.880 | 0.880 | 0.907 |
| 2 | miR-374c-5p | 0.795 | 0.823 | 0.852 | 0.873 | 0.892 | 0.892 | 0.910 |
| 3 | miR-324-5p | 0.747 | 0.796 | 0.829 | 0.869 | 0.892 | 0.892 | 0.910 |
| 4 | miR-24-3p | 0.707 | 0.796 | 0.827 | 0.847 | 0.887 | 0.887 | 0.910 |
| 5 | miR-133a-3p | 0.660 | 0.765 | 0.803 | 0.839 | 0.880 | 0.880 | 0.907 |
| 6 | miR-125b-5p | 0.660 | 0.749 | 0.805 | 0.836 | 0.880 | 0.880 | 0.910 |
| 7 | miR-497-5p | 0.660 | 0.746 | 0.798 | 0.834 | 0.880 | 0.880 | 0.907 |
| 8 | miR-19b-3p | 0.657 | 0.750 | 0.798 | 0.836 | 0.880 | 0.880 | 0.907 |

In one example, the groups of 4 miRNA are, but are not limited to, the following groups: miR-133a-3p, miR-497-5p, miR-24-3p, miR-125b-5p; miR-133a-3p, miR-497-5p, miR-24-3p, miR-377-3p; miR-133a-3p, miR-497-5p, miR-24-3p, miR-374c-5p; miR-133a-3p, miR-497-5p, miR-24-3p, miR-324-5p; miR-133a-3p, miR-497-5p, miR-24-3p, miR-19b-3p; miR-133a-3p, miR-497-5p, miR-125b-5p, miR-377-3p; miR-133a-3p, miR-497-5p, miR-125b-5p, miR-374c-5p; miR-133a-3p, miR-497-5p, miR-125b-5p, miR-324-5p; miR-133a-3p, miR-497-5p, miR-125b-5p, miR-19b-3p; miR-133a-3p, miR-497-5p, miR-377-3p, miR-374c-5p; miR-133a-3p, miR-497-5p, miR-377-3p, miR-324-5p; miR-133a-3p, miR-497-5p, miR-377-3p, miR-19b-3p; miR-133a-3p, miR-497-5p, miR-374c-5p, miR-324-5p; miR-133a-3p, miR-497-5p, miR-374c-5p, miR-19b-3p; miR-133a-3p, miR-497-5p, miR-324-5p, miR-19b-3p; miR-133a-3p, miR-24-3p, miR-125b-5p, miR-377-3p; miR-133a-3p, miR-24-3p, miR-125b-5p, miR-374c-5p; miR-133a-3p, miR-24-3p, miR-125b-5p, miR-324-5p; miR-133a-3p, miR-24-3p, miR-125b-5p, miR-19b-3p; miR-133a-3p, miR-24-3p, miR-377-3p, miR-374c-5p; miR-133a-3p, miR-24-3p, miR-377-3p, miR-324-5p; miR-133a-3p, miR-24-3p, miR-377-3p, miR-19b-3p; miR-133a-3p, miR-24-3p, miR-374c-5p, miR-324-5p; miR-133a-3p, miR-24-3p, miR-374c-5p, miR-19b-3p; miR-133a-3p, miR-24-3p, miR-324-5p, miR-19b-3p; miR-133a-3p, miR-125b-5p, miR-377-3p, miR-374c-5p; miR-133a-3p, miR-125b-5p, miR-377-3p, miR-324-5p; miR-133a-3p, miR-125b-5p, miR-377-3p, miR-19b-3p; miR-133a-3p, miR-125b-5p, miR-374c-5p, miR-324-5p; miR-133a-3p, miR-125b-5p, miR-374c-5p, miR-19b-3p; miR-133a-3p, miR-125b-5p, miR-324-5p, miR-19b-3p; miR-133a-3p, miR-377-3p, miR-374c-5p, miR-324-5p; miR-133a-3p, miR-377-3p, miR-374c-5p, miR-19b-3p; miR-133a-3p, miR-377-3p, miR-324-5p, miR-19b-3p; miR-133a-3p, miR-374c-5p, miR-324-5p, miR-19b-3p; miR-497-5p, miR-24-3p, miR-125b-5p, miR-377-3p; miR-497-5p, miR-24-3p, miR-125b-5p, miR-374c-5p; miR-497-5p, miR-24-3p, miR-125b-5p, miR-324-5p; miR-497-5p, miR-24-3p, miR-125b-5p, miR-19b-3p; miR-497-5p, miR-24-3p, miR-377-3p, miR-374c-5p; miR-497-5p, miR-24-3p, miR-377-3p, miR-324-5p; miR-497-5p, miR-24-3p, miR-377-3p, miR-19b-3p; miR-497-5p, miR-24-3p, miR-374c-5p, miR-324-5p; miR-497-5p, miR-24-3p, miR-374c-5p, miR-19b-3p; miR-497-5p, miR-24-3p, miR-324-5p, miR-19b-3p; miR-497-5p, miR-125b-5p, miR-377-3p, miR-374c-5p; miR-497-5p, miR-125b-5p, miR-377-3p, miR-324-5p; miR-497-5p, miR-125b-5p, miR-377-3p, miR-19b-3p; miR-497-5p, miR-125b-5p, miR-374c-5p, miR-324-5p; miR-497-5p, miR-125b-5p, miR-374c-5p, miR-19b-3p; miR-497-5p, miR-125b-5p, miR-324-5p, miR-19b-3p; miR-497-5p, miR-377-3p, miR-374c-5p, miR-324-5p; miR-497-5p, miR-377-3p, miR-374c-5p, miR-19b-3p; miR-497-5p, miR-377-3p, miR-324-5p, miR-19b-3p; miR-497-5p, miR-374c-5p, miR-324-5p, miR-19b-3p; miR-24-3p, miR-125b-5p, miR-377-3p, miR-374c-5p; miR-24-3p, miR-125b-5p, miR-377-3p, miR-324-5p; miR-24-3p, miR-125b-5p, miR-377-3p, miR-19b-3p; miR-24-3p, miR-125b-5p, miR-374c-5p, miR-324-5p; miR-24-3p, miR-125b-5p, miR-374c-5p, miR-19b-3p; miR-24-3p, miR-125b-5p, miR-324-5p, miR-19b-3p; miR-24-3p, miR-377-3p, miR-374c-5p, miR-324-5p; miR-24-3p, miR-377-3p, miR-374c-5p, miR-19b-3p; miR-24-3p, miR-377-3p, miR-324-5p, miR-19b-3p; miR-24-3p, miR-374c-5p, miR-324-5p, miR-19b-3p; miR-125b-5p, miR-377-3p, miR-374c-5p, miR-324-5p; miR-125b-5p, miR-377-3p, miR-374c-5p, miR-19b-3p; miR-125b-5p, miR-377-3p, miR-324-5p, miR-19b-3p; miR-125b-5p, miR-374c-5p, miR-324-5p, miR-19b-3p; and miR-377-3p, miR-374c-5p, miR-324-5p, miR-19b-3p.

In addition to the groups of 4 miRNA listed above, in one example, 1, 2, 3, 4, 5, 6, 7, 8 or more additional miRNA are added to the panel. In another example, the method disclosed herein comprises detecting at least 3, at least 4, at least 5, at least 6, at least 7, or at least 8 miRNA markers.

These additional miRNAs can be added to the group, so long as they are not already present in the group of 4 as previously described.

Thus, in one example, there is disclosed a method for determining whether a subject is suffering from, or is at risk of, developing breast cancer. In one example, the method comprises detecting differential expression levels of at least two or more miRNA markers from a biological sample obtained from the subject. In one example, the method comprises detecting differential expression levels of at least two, at least 3, at least 4, at least 5, at least 6, at least 7, at least 8, or more miRNA markers from a biological sample obtained from the subject. In yet another example, the method disclosed herein comprises detecting 3, 4, 5, 6, 7, 8, or more miRNA.

In one example, the groups of 3 miRNA are, but are not limited to, the following groups: miR-133a-3p, miR-497-5p, miR-24-3p; miR-133a-3p, miR-497-5p, miR-125b-5p; miR-133a-3p, miR-497-5p, miR-377-3p; miR-133a-3p, miR-497-5p, miR-374c-5p; miR-133a-3p, miR-497-5p, miR-324-5p; miR-133a-3p, miR-497-5p, miR-19b-3p; miR-133a-3p, miR-24-3p, miR-125b-5p; miR-133a-3p, miR-24-3p, miR-377-3p; miR-133a-3p, miR-24-3p, miR-374c-5p; miR-133a-3p, miR-24-3p, miR-324-5p; miR-133a-3p, miR-24-3p, miR-19b-3p; miR-133a-3p, miR-125b-5p, miR-377-3p; miR-133a-3p, miR-125b-5p, miR-374c-5p; miR-133a-3p, miR-125b-5p, miR-324-5p; miR-133a-3p, miR-125b-5p, miR-19b-3p; miR-133a-3p, miR-377-3p, miR-374c-5p; miR-133a-3p, miR-377-3p, miR-324-5p; miR-133a-3p, miR-377-3p, miR-19b-3p; miR-133a-3p, miR-374c-5p, miR-324-5p; miR-133a-3p, miR-374c-5p, miR-19b-3p; miR-133a-3p, miR-324-5p, miR-19b-3p; miR-497-5p, miR-24-3p, miR-125b-5p; miR-497-5p, miR-24-3p, miR-377-3p; miR-497-5p, miR-24-3p, miR-374c-5p; miR-497-5p, miR-24-3p, miR-324-5p; miR-497-5p, miR-24-3p, miR-19b-3p; miR-497-5p, miR-125b-5p, miR-377-3p; miR-497-5p, miR-125b-5p, miR-374c-5p; miR-497-5p, miR-125b-5p, miR-324-5p; miR-497-5p, miR-125b-5p, miR-19b-3p; miR-497-5p, miR-377-3p, miR-374c-5p; miR-497-5p, miR-377-3p, miR-324-5p; miR-497-5p, miR-377-3p, miR-19b-3p; miR-497-5p, miR-374c-5p, miR-324-5p; miR-497-5p, miR-374c-5p, miR-19b-3p; miR-497-5p, miR-324-5p, miR-19b-3p; miR-24-3p, miR-125b-5p, miR-377-3p; miR-24-3p, miR-125b-5p, miR-374c-5p; miR-24-3p, miR-125b-5p, miR-324-5p; miR-24-3p, miR-125b-5p, miR-19b-3p; miR-24-3p, miR-377-3p, miR-374c-5p; miR-24-3p, miR-377-3p, miR-324-5p; miR-24-3p, miR-377-3p, miR-19b-3p; miR-24-3p, miR-374c-5p, miR-324-5p; miR-24-3p, miR-374c-5p, miR-19b-3p; miR-24-3p, miR-324-5p, miR-19b-3p; miR-125b-5p, miR-377-3p, miR-374c-5p; miR-125b-5p, miR-377-3p, miR-324-5p; miR-125b-5p, miR-377-3p, miR-19b-3p; miR-125b-5p, miR-374c-5p, miR-324-5p; miR-125b-5p, miR-374c-5p, miR-19b-3p; miR-125b-5p, miR-324-5p, miR-19b-3p; miR-377-3p, miR-374c-5p, miR-324-5p; miR-377-3p, miR-374c-5p, miR-19b-3p; miR-377-3p, miR-324-5p, miR-19b-3p; and miR-374c-5p, miR-324-5p, miR-19b-3p.

In one example, the groups of 5 miRNA are, but are not limited to, the following groups: miR-133a-3p, miR-497-5p, miR-24-3p, miR-125b-5p, miR-377-3p; miR-133a-3p, miR-497-5p, miR-24-3p, miR-125b-5p, miR-374c-5p; miR-133a-3p, miR-497-5p, miR-24-3p, miR-125b-5p, miR-324-5p; miR-133a-3p, miR-497-5p, miR-24-3p, miR-125b-5p, miR-19b-3p; miR-133a-3p, miR-497-5p, miR-24-3p, miR-377-3p, miR-374c-5p; miR-133a-3p, miR-497-5p, miR-24-3p, miR-377-3p, miR-324-5p; miR-133a-3p, miR-497-5p, miR-24-3p, miR-377-3p, miR-19b-3p; miR-133a-3p, miR-497-5p, miR-24-3p, miR-374c-5p, miR-324-5p; miR-133a-3p, miR-497-5p, miR-24-3p, miR-374c-5p, miR-19b-3p; miR-133a-3p, miR-497-5p, miR-24-3p, miR-324-5p, miR-19b-3p; miR-133a-3p, miR-497-5p, miR-125b-5p, miR-377-3p, miR-374c-5p; miR-133a-3p, miR-497-5p, miR-125b-5p, miR-377-3p, miR-324-5p; miR-133a-3p, miR-497-5p, miR-125b-5p, miR-377-3p, miR-19b-3p; miR-133a-3p, miR-497-5p, miR-125b-5p, miR-374c-5p, miR-324-5p; miR-133a-3p, miR-497-5p, miR-125b-5p, miR-374c-5p, miR-19b-3p; miR-133a-3p, miR-497-5p, miR-125b-5p, miR-324-5p, miR-19b-3p; miR-133a-3p, miR-497-5p, miR-377-3p, miR-374c-5p, miR-324-5p; miR-133a-3p, miR-497-5p, miR-377-3p, miR-374c-5p, miR-19b-3p; miR-133a-3p, miR-497-5p, miR-377-3p, miR-324-5p, miR-19b-3p; miR-133a-3p, miR-497-5p, miR-374c-5p, miR-324-5p, miR-19b-3p; miR-133a-3p, miR-24-3p, miR-125b-5p, miR-377-3p, miR-374c-5p; miR-133a-3p, miR-24-3p, miR-125b-5p, miR-377-3p, miR-324-5p; miR-133a-3p, miR-24-3p, miR-125b-5p, miR-377-3p, miR-19b-3p; miR-133a-3p, miR-24-3p, miR-125b-5p, miR-374c-5p, miR-324-5p; miR-133a-3p, miR-24-3p, miR-125b-5p, miR-374c-5p, miR-19b-3p; miR-133a-3p, miR-24-3p, miR-125b-5p, miR-324-5p, miR-19b-3p; miR-133a-3p, miR-24-3p, miR-377-3p, miR-374c-5p, miR-324-5p; miR-133a-3p, miR-24-3p, miR-377-3p, miR-374c-5p, miR-19b-3p; miR-133a-3p, miR-24-3p, miR-377-3p, miR-324-5p, miR-19b-3p; miR-133a-3p, miR-24-3p, miR-374c-5p, miR-324-5p, miR-19b-3p; miR-133a-3p, miR-125b-5p, miR-377-3p, miR-374c-5p, miR-324-5p; miR-133a-3p, miR-125b-5p, miR-377-3p, miR-374c-5p, miR-19b-3p; miR-133a-3p, miR-125b-5p, miR-377-3p, miR-324-5p, miR-19b-3p; miR-133a-3p, miR-125b-5p, miR-374c-5p, miR-324-5p, miR-19b-3p; miR-133a-3p, miR-377-3p, miR-374c-5p, miR-324-5p, miR-19b-3p; miR-497-5p, miR-24-3p, miR-125b-5p, miR-377-3p, miR-374c-5p; miR-497-5p, miR-24-3p, miR-125b-5p, miR-377-3p, miR-324-5p; miR-497-5p, miR-24-3p, miR-125b-5p, miR-377-3p, miR-19b-3p; miR-497-5p, miR-24-3p, miR-125b-5p, miR-374c-5p, miR-324-5p; miR-497-5p, miR-24-3p, miR-125b-5p, miR-374c-5p, miR-19b-3p; miR-497-5p, miR-24-3p, miR-125b-5p, miR-324-5p, miR-19b-3p; miR-497-5p, miR-24-3p, miR-377-3p, miR-374c-5p, miR-324-5p; miR-497-5p, miR-24-3p, miR-377-3p, miR-374c-5p, miR-19b-3p; miR-497-5p, miR-24-3p, miR-377-3p, miR-324-5p, miR-19b-3p; miR-497-5p, miR-24-3p, miR-374c-5p, miR-324-5p, miR-19b-3p; miR-497-5p, miR-125b-5p, miR-377-3p, miR-374c-5p, miR-324-5p; miR-497-5p, miR-125b-5p, miR-377-3p, miR-374c-5p, miR-19b-3p; miR-497-5p, miR-125b-5p, miR-377-3p, miR-324-5p, miR-19b-3p; miR-497-5p, miR-125b-5p, miR-374c-5p, miR-324-5p, miR-19b-3p; miR-497-5p, miR-377-3p, miR-374c-5p, miR-324-5p, miR-19b-3p; miR-24-3p, miR-125b-5p, miR-377-3p, miR-374c-5p, miR-324-5p; miR-24-3p, miR-125b-5p, miR-377-3p, miR-374c-5p, miR-19b-3p; miR-24-3p, miR-125b-5p, miR-377-3p, miR-324-5p, miR-19b-3p; miR-24-3p, miR-125b-5p, miR-374c-5p, miR-324-5p, miR-19b-3p; miR-24-3p, miR-377-3p, miR-374c-5p, miR-324-5p, miR-19b-3p; and miR-125b-5p, miR-377-3p, miR-374c-5p, miR-324-5p, miR-19b-3p.

In one example, the groups of 6 miRNA are, but are not limited to, the following groups: miR-133a-3p, miR-497-5p, miR-24-3p, miR-125b-5p, miR-377-3p, miR-374c-5p; miR-133a-3p, miR-497-5p, miR-24-3p, miR-125b-5p, miR-377-3p, miR-324-5p; miR-133a-3p, miR-497-5p, miR-24-3p, miR-125b-5p, miR-377-3p, miR-19b-3p; miR-133a-3p, miR-497-5p, miR-24-3p, miR-125b-5p, miR-374c-5p, miR-324-5p; miR-133a-3p, miR-497-5p, miR-24-3p, miR-125b-5p, miR-374c-5p, miR-19b-3p; miR-133a-3p, miR-497-5p, miR-24-3p, miR-125b-5p, miR-324-5p, miR-19b-3p; miR-133a-3p, miR-497-5p, miR-24-3p, miR-377-3p, miR-374c-5p, miR-324-5p; miR-133a-3p, miR-497-5p, miR-24-3p, miR-377-3p, miR-374c-5p, miR-19b-3p; miR-133a-3p, miR-497-5p, miR-24-3p, miR-377-3p, miR-324-5p, miR-19b-3p; miR-133a-3p, miR-497-5p, miR-24-3p, miR-374c-5p, miR-324-5p, miR-19b-3p; miR-133a-3p, miR-497-5p, miR-125b-5p, miR-377-3p, miR-374c-5p, miR-324-5p; miR-133a-3p, miR-497-5p, miR-125b-5p, miR-377-3p, miR-374c-5p, miR-19b-3p; miR-133a-3p, miR-497-5p, miR-125b-5p, miR-377-3p, miR-324-5p, miR-19b-3p; miR-133a-3p, miR-497-5p, miR-125b-5p, miR-374c-5p, miR-324-5p, miR-19b-3p; miR-133a-3p, miR-497-5p, miR-377-3p, miR-374c-5p, miR-324-5p, miR-19b-3p; miR-133a-3p, miR-24-3p, miR-125b-5p, miR-377-3p, miR-374c-5p, miR-324-5p; miR-133a-3p, miR-24-3p, miR-125b-5p, miR-377-3p, miR-374c-5p, miR-19b-3p; miR-133a-3p, miR-24-3p, miR-125b-5p, miR-377-3p, miR-324-5p, miR-19b-3p; miR-133a-3p, miR-24-3p, miR-125b-5p, miR-374c-5p, miR-324-5p, miR-19b-3p; miR-133a-3p, miR-24-3p, miR-377-3p, miR-374c-5p, miR-324-5p, miR-19b-3p; miR-133a-3p, miR-125b-5p, miR-377-3p, miR-374c-5p, miR-324-5p, miR-19b-3p; miR-497-5p, miR-24-3p, miR-125b-5p, miR-377-3p, miR-374c-5p, miR-324-5p; miR-497-5p, miR-24-3p, miR-125b-5p, miR-377-3p, miR-374c-5p, miR-19b-3p; miR-497-5p, miR-24-3p, miR-125b-5p, miR-377-3p, miR-324-5p, miR-19b-3p; miR-497-5p, miR-24-3p, miR-125b-5p, miR-374c-5p, miR-324-5p, miR-19b-3p; miR-497-5p, miR-24-3p, miR-377-3p, miR-374c-5p, miR-324-5p, miR-19b-3p; miR-497-5p, miR-125b-5p, miR-377-3p, miR-374c-5p, miR-324-5p, miR-19b-3p; and miR-24-3p, miR-125b-5p, miR-377-3p, miR-374c-5p, miR-324-5p, miR-19b-3p.

In one example, the groups of 7 miRNA are, but are not limited to, the following groups: miR-133a-3p, miR-497-5p, miR-24-3p, miR-125b-5p, miR-377-3p, miR-374c-5p, miR-324-5p; miR-133a-3p, miR-497-5p, miR-24-3p, miR-125b-5p, miR-377-3p, miR-374c-5p, miR-19b-3p; miR-133a-3p, miR-497-5p, miR-24-3p, miR-125b-5p, miR-377-3p, miR-324-5p, miR-19b-3p; miR-133a-3p, miR-497-5p, miR-24-3p, miR-125b-5p, miR-374c-5p, miR-324-5p, miR-19b-3p; miR-133a-3p, miR-497-5p, miR-24-3p, miR-377-3p, miR-374c-5p, miR-324-5p, miR-19b-3p; miR-133a-3p, miR-497-5p, miR-125b-5p, miR-377-3p, miR-374c-5p, miR-324-5p, miR-19b-3p; miR-133a-3p, miR-24-3p, miR-125b-5p, miR-377-3p, miR-374c-5p, miR-324-5p, miR-19b-3p; and miR-497-5p, miR-24-3p, miR-125b-5p, miR-377-3p, miR-374c-5p, miR-324-5p, miR-19b-3p.

In one example, the miRNAs are selected from miR-133a-3p, miR-497-5p, miR-24-3p, miR-125b-5p, miR-377-3p, miR-374c-5p, miR-324-5p and miR-19b-3p, and the differential expression level is compared with that of a cancer-free subject.

In on example, the differential expression is based on up- and/or downregulation of the miRNA, wherein, if present, the following miRNA are upregulated in a subject suffering from, or at risk of developing a breast cancer: miR-133a-3p, miR-497-5p, mir-24-3p, and miR-125b-5p.

In another example, the differential expression is based on up- and/or downregulation of the miRNA, wherein, if present, the following miRNA are downregulated in a subject suffering from, or at risk of developing breast cancer: miR-377-3p, miR-374c-5p, miR-324-5p and miR-19b-3p.

In another example, the method disclosed herein is a method of determining whether a subject is suffering from, or is at risk of developing, breast cancer, the method comprising: i. detecting the presence of miRNA in a bodily fluid sample obtained from the subject; ii. measuring the expression level of at least two miRNAs in the bodily fluid sample; and iii. using a prediction algorithm score based on the differential expression level of the miRNAs measured previously to predict the probability of the subject to suffer from or develop breast cancer, wherein the at least two or more miRNA markers are selected from miR-133a-3p, miR-497-5p, miR-24-3p, miR-125b-5p, miR-377-3p, miR-374c-5p, miR-324-5p and miR-19b-3p, and wherein the differential expression of miR-377-3p, miR-374c-5p, miR-324-5p and miR-19b-3p, if present, are downregulated, as compared to a control, or wherein the differential expression of miR-133a-3p, miR-497-5p, mir-24-3p, and miR-125b-5p, if present, are upregulated, as compared to a control, and determining the subject to suffer from breast cancer or to be at risk of developing breast cancer, and treating the subject determined to suffer from breast cancer or determined to be at risk of developing breast cancer with an anti-breast cancer compound, wherein the control for comparing the expression level of the at least two miRNAs referred to in step ii) is a breast cancer-free subject.

In another example, there is disclosed a method of treating breast cancer. In yet another example, the method of treating breast cancer comprises i) detecting the presence of miRNA in a bodily fluid sample obtained from the subject; ii) measuring the expression level of at least two miRNA in the bodily fluid sample; and iii) using a prediction algorithm score based on the differential expression level of the miRNAs measured previously to predict the probability of the subject to suffer from or develop breast cancer, wherein the at least two or more miRNA markers are selected from miR-133a-3p, miR-497-5p, miR-24-3p, miR-125b-5p, miR-377-3p, miR-374c-5p, miR-324-5p and miR-19b-3p, and wherein the differential expression of miR-377-3p, miR-374c-5p, miR-324-5p and miR-19b-3p, if present, are downregulated, as compared to a control, or wherein the differential expression of miR-133a-3p, miR-497-5p, mir-24-3p, and miR-125b-5p, if present, are upregulated, as compared to a control, and determining the subject to suffer from breast cancer or to be at risk of developing breast cancer, wherein the control for comparing the expression level of the at least two miRNAs referred to in step ii) is a breast cancer-free subject.

In yet another example, there is disclosed a method of treating breast cancer comprises i) detecting the presence of miRNA in a bodily fluid sample obtained from the subject; ii) measuring the expression level of at least two miRNA in the bodily fluid sample; and iii) using a prediction algorithm score based on the differential expression level of the miRNAs measured previously to predict the probability of the subject to suffer from or develop breast cancer, wherein the at least two or more miRNA markers are selected from miR-133a-3p, miR-497-5p, miR-24-3p, miR-125b-5p, miR-377-3p, miR-374c-5p, miR-324-5p and miR-19b-3p, and wherein the differential expression of miR-377-3p, miR-374c-5p, miR-324-5p and miR-19b-3p, if present, are downregulated, as compared to a control, or wherein the differential expression of miR-133a-3p, miR-497-5p, mir-24-3p, and miR-125b-5p, if present, are upregulated, as compared to a control, and determining the subject to suffer from breast cancer or to be at risk of developing breast cancer, and treating the subject determined to suffer from breast cancer or determined to be at risk of developing breast cancer with an anti-breast cancer compound, wherein the control for comparing the expression level of the at least two miRNAs referred to in step ii) is a breast cancer-free subject.

For the performance comparison of the prediction model in a Singaporean Chinese cohort, the present model was able to achieve a better classification (AUC of 0.973) than most of the existing miRNA panels, and was based on a large size of patient samples in both training and the multiple validation phases.

Another observation from previous studies is that there is a lack of strong overlap of miRNAs between studies which could possibly be attributed to differences between studies in sample type (whole blood, plasma or serum), timing of blood collection (before or after surgery), technology platform (microarray, RT-PCR or next-generation sequencing), study design and differences in data analysis. Hence, this indicates that for biomarker discovery research, having multiple validation cohorts is beneficial in order to verify the biomarker signature.

In one example, if a subject is determined to be suffering from or at the risk of developing breast cancer, then the subject is treated against breast cancer or the onset of breast cancer with any one or more of the following anti-breast cancer treatments: surgery, radiation therapy, chemotherapy, hormone therapy, targeted therapy, immunotherapy or one or more anti-breast cancer compounds, when the subject is determined to have breast cancer or determined to be at a risk of developing breast cancer. In another example, the subject is treated using the standard of care available for treating the type or stage of cancer that the subject is determined to have.

In one example, the method disclosed herein determines the subject to suffer from cancer, and the cancer is determined to be an early stage (i.e. a cancer of stage 0, I, or II) or a late-stage cancer (i.e. a cancer of stage III or IV). In one example, the determination of the cancer stage is performed using alternative methods known in the art, such as, but not limited to, histological or immunohistological analyses. In another example, the stage of the cancer is unknown.

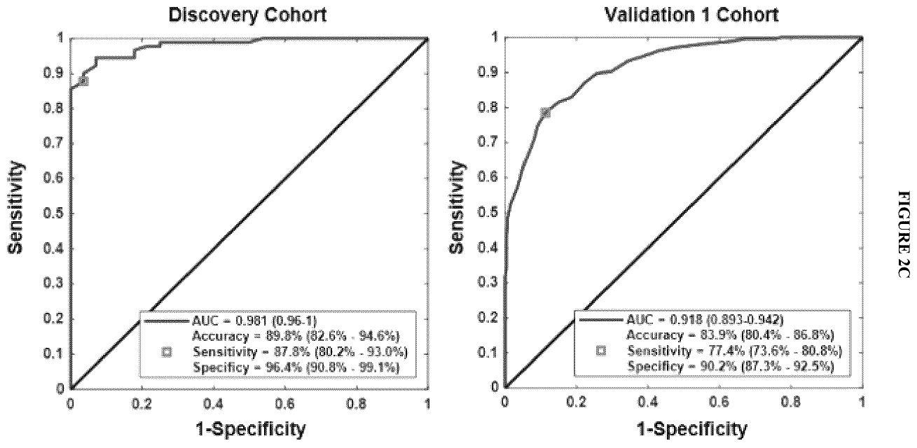

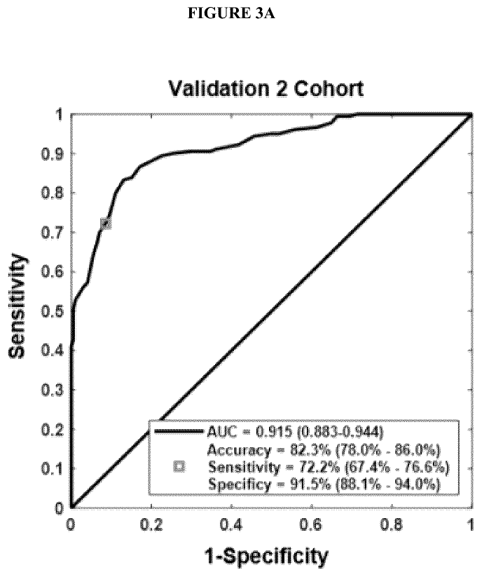

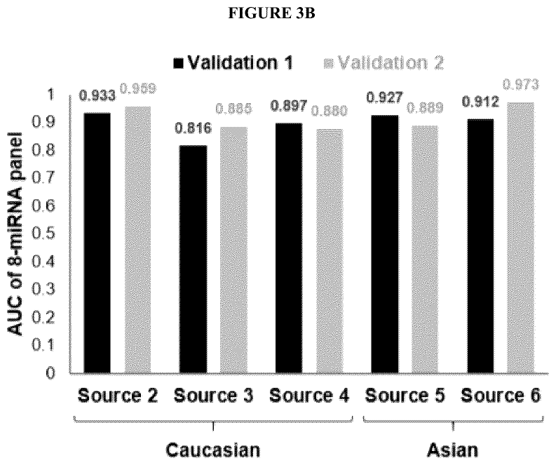

As shown in, for example, in FIG. 3C, this figure shows the receiver operating characteristic (ROC) curves for performance of an eight-miRNA biomarker panel as disclosed herein in predicting early (stages 0, I and II) and late (stages III and IV) breast cancer in the Validation 2 cohort. This data demonstrates that the eight-miRNA panel as disclosed herein is able to detect cancer of all stages (even early-stage cancer) with good performance (AUC=0.831 for stage 0; AUC=0.916 for stages 0, I and II; AUC=0.953 for later stages (Stage III and IV). This reflects the ability of the method as disclosed herein to detect the presence of cancer at different stages. This applies even in early stages of cancer, where the tumour is small or potentially not visible. As expected, late-stage cancers, where the tumour is larger, are detected with the highest AUC. With better outcome for treatment of early-stage cancer, the ability to diagnose breast cancer at an earlier stage, especially where the tumour is relatively small and difficult to detect via imaging or ultrasound, would have significant benefits to patients.

The information disclosed herein utilized qRT-PCR for miRNA profiling, since qRT-PCR is deemed as the standard for nucleic acid quantification due to the sensitivity and specificity of the method. In the analysis, the copy number of miRNA targets was used instead of the relative expression of each miRNA. In addition, since qRT-PCR is commonly utilized in various multigene prognostic assays including Oncotype DX, Breast Cancer Index, and EndoPredict, this makes the miRNA-based breast cancer prediction model disclosed herein readily translatable as a molecular diagnostic assay for clinical use.

Apart from miRNA biomarkers, there are other efforts assessing alternative blood-based bioanalytes for breast cancer detection, such as the CancerSEEK study and the Circulating Cell-Free Genome Atlas (CCGA) study. CancerSEEK is a pan-cancer blood test intended for the identification of eight cancer types including breast cancer, by evaluating mutations in 16 genes from cell-free DNA (cfDNA) and the expression of eight protein biomarkers using multiplex PCR and immunoassays respectively. Similarly, the CCGA study which is an on-going prospective longitudinal cohort study that has enrolled approximately 15,000 study participants, also aims to develop a multi-cancer detection blood test by profiling cfDNA using sequencing-based methods. Although these assays have been tested to detect different cancer types and stages, their performance for identifying breast cancer, especially in the early stages, is still under par. For the CancerSEEK test, the median detection sensitivity for breast cancer was 33% as compared to 98% for ovarian cancer, whereas the median detection sensitivity for stage I of all cancer types was only 43% as compared to 78% for stage III cancers. Moreover, the tests developed by the CCGA study were poor in identifying various breast cancer molecular subtypes with sensitivities below 60%. In contrast, the miRNA-based model disclosed herein showed superior discrimination performance, even for differentiating between heathy controls and those at pre-malignant stages (stage 0) with the AUC, accuracy, sensitivity and specificity of 0.831, 87.4%, 52.2% and 91.5% respectively. In addition, the AUC and sensitivity increased to 0.916 and 71.4% respectively for the detection of the pre-malignant stage and early-stage breast cancers (stages 0-II).

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### EXAMPLES

### Example 1: Discovery and Validation of Significant Differentially Regulated miRNAs

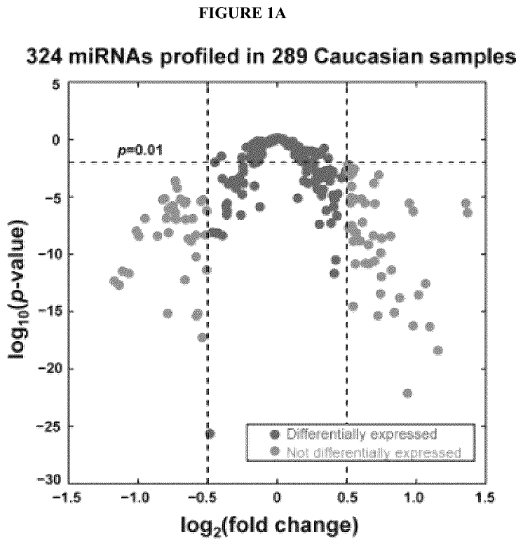

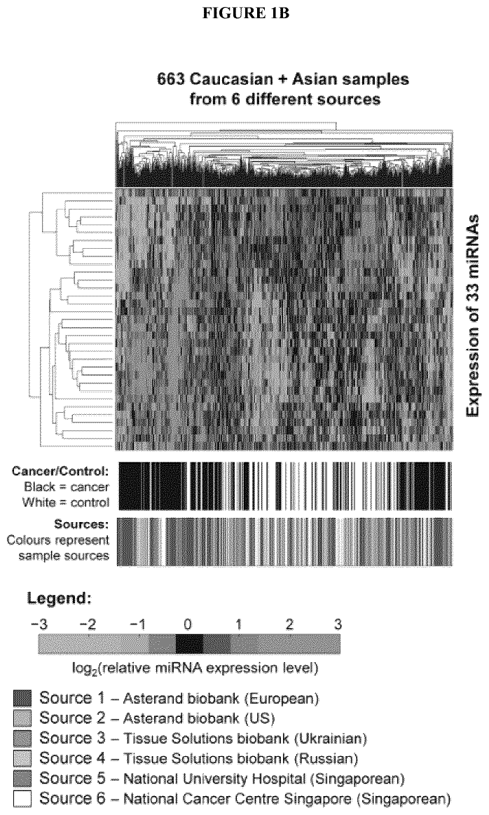

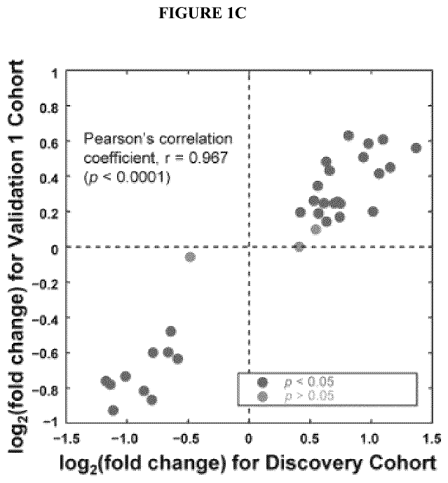

The expression levels of 324 miRNAs, which have been previously detected with high confidence in human serum, were quantified in the Discovery Cohort of 289 Caucasian samples (183 breast cancer samples and 106 non-cancer controls). All samples in this cohort were obtained from a single source as shown in Table 1. MiRNAs that were significantly differentially expressed between breast cancer cases and non-cancer controls were identified by p-value of unpaired Student's t-test and fold change in expression. A total of 86 miRNAs that were differentially expressed between cancer cases and non-cancer controls with log 2 (fold change) more than 0.5 or less than −0.5 and p-value <0.01 were identified (FIG. 1A).

The ability of these 86 differentially expressed miRNAs to differentiate between breast cancer cases and non-cancer controls was also assessed using AUC analysis. Out of these 86 differentially expressed miRNAs, 33 miRNAs had AUC >0.5 and were selected for validation in a mixed Caucasian-Asian cohort (Validation 1). This cohort comprised of 374 samples (177 breast cancer cases and 197 non-cancer controls) from five different sources (three Caucasian and two Asian populations) as shown in Table 1.

Unsupervised hierarchical clustering based on the differential expressions of the 33 top-ranked miRNA biomarker candidates was carried out on the combined Discovery and Validation 1 cohort (663 samples comprising 360 breast cancer cases and 303 non-cancer controls). The cancer samples and the non-cancer samples were partially separated after clustering based on differential expression of these 33 miRNAs (FIG. 1B). Additionally, samples from the same sources were not clustered together based on their expression of these 33 miRNAs (FIG. 1B).

The log 2 (fold change) calculated for these 33 miRNA biomarker candidates in the Discovery cohort and Validation 1 cohort were compared and had a Pearson's correlation coefficient, r=0.967 (p<0.0001). Out of the 33 biomarker candidates identified from the Discovery cohort, 30 miRNAs were differentially expressed in breast cancer cases compared to non-cancer controls (p<0.05 by unpaired t-test) in the Validation 1 cohort. All 33 biomarker candidates were differentially regulated in the Validation 1 cohort, with consistent log 2 (fold change) values between the two cohorts (FIG. 1C). Three miRNA biomarker candidates that showed non-significant differential expressions (p >0.05) in Validation 1 cohort were excluded from the subsequent analysis, while the remaining 30 miRNA biomarker candidates were used for the biomarker panel optimization phase.

### Example 2: Optimization of miRNA Biomarker Panels

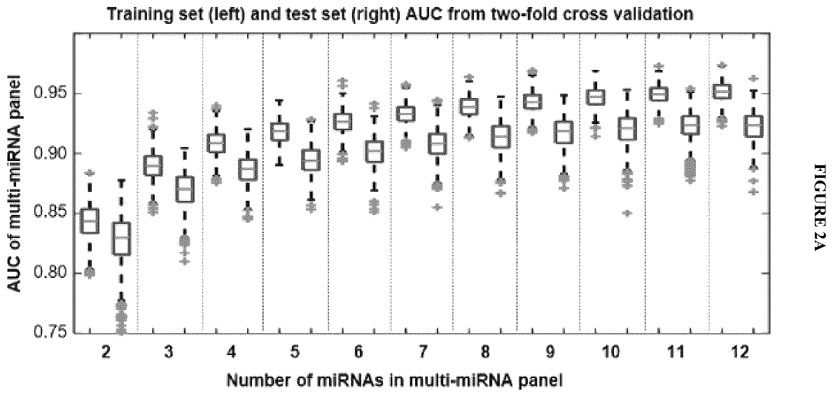

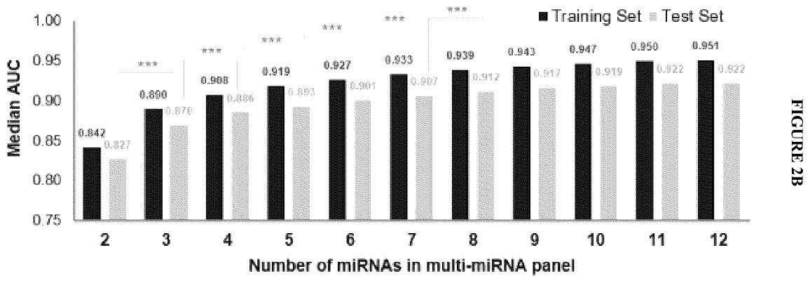

To identify an optimal panel with good performance while balancing the number of miRNAs included for practicality of clinical testing, multi-miRNA panels were assessed. The best-performing multi-miRNA panel comprising between two to twelve miRNAs were formed from the 30 validated miRNA biomarker candidates using a two-fold cross-validation procedure that incorporated a feature selection algorithm (SFSS). AUC of miRNA panel performance in the training and test group was calculated for 200 iterations of cross-validation with multi-miRNA panels comprising two to twelve miRNAs (FIG. 2A). The median AUC for breast cancer prediction from 200 iterations of training and testing was calculated for each set of cross-validation experiments comprising two to twelve miRNAs (FIG. 2B). The median AUC increased significantly (p<0.001) with increasing number of miRNAs in the biomarker panels that consisted of two to eight miRNAs, until it reached a plateau after the inclusion of eight miRNAs on the panel (FIG. 2B); hence indicating that eight miRNAs is the optimal number of biomarkers to be included on the panel. The addition of more miRNAs did not lead to a statistically significant increase in AUC. The optimal eight-miRNA biomarker panel with the highest AUCs of 0.981 and 0.918 in the Discovery and Validation 1 cohorts, respectively, was chosen for further validation (FIG. 2C). This optimal panel included miR-133a-3p, miR-497-5p, mir-24-3p, and miR-125b-5p, which were upregulated in breast cancer cases compared to controls, and miR-377-3p, miR-374c-5p, miR-324-5p and miR-19b-3p, which were downregulated in breast cancer cases as compared to controls. At the point of maximum classification accuracy, sensitivity and specificity were 87.8% (95% CI, 80.2%-93.0%) and 96.4% (95% CI, 90.8%-99.1%) in the Discovery Cohort, and 77.4% (95% CI, 73.6%-80.8%) and 90.2% (95% CI, 87.3%-92.5%) in the Validation 1 Cohort (FIG. 2C).

### Example 3: Validation of Optimal Eight-miRNA Biomarker Panel Signature

Validation of the optimized eight-miRNA biomarker panel signature was carried out in the Validation 2 cohort which comprised of 379 samples (180 breast cancer and 199 non-cancer samples). The AUC of the eight-miRNA biomarker panel in classifying breast cancer and non-cancer samples was 0.915 (95% CI, 0.883-0.944) (FIG. 3A). At the point of maximum classification accuracy, sensitivity was 72.2% (95% CI, 67.4%-76.6%) with a specificity of 91.5% (95% CI, 88.1%-94.0%). When the Validation 1 and 2 cohorts were separated by sample source, the eight-miRNA biomarker panel had AUC ranging from 0.816 to 0.973 (FIG. 3B). Performance was comparable between Caucasian and Asian sample sources (FIG. 3B) and for early stage (stages 0, I and II) and late stage (stages III and IV) breast cancers (FIG. 3C). The performance of the present miRNA-based prediction model for the initial discovery and two validation phases were consistent, as demonstrated by their respective AUCs of 0.981, 0.918 and 0.915. Likewise, when the validation phases 1 and 2 were analysed based on the sub-cohorts obtained from different sample sources, the range of AUCs generated in these sub-cohorts for both phases were comparable, ranging from 0.816 to 0.933 in phase 1 and from 0.880 to 0.973 in phase 2. These results highlight the high reproducibility and accuracy of the model in differentiating breast cancer cases from non-cancer controls for both Caucasians and Asians, suggesting its potential universal usability for various ethnicities.

Among the eight miRNAs in the miRNA panel disclosed herein, miR-133a-3p, miR-497-5p, mir-24-3p, and miR-125b-5p were found to be upregulated, whereas miR-377-3p, miR-374c-5p, miR-324-5p and miR-19b-3p were found to be downregulated in breast cancer cases as compared to controls. For example, both miR-24-3p and miR-125b-5p have been identified as potential breast cancer biomarkers for the early detection, prognosis, or prediction of recurrence. Among the eight miRNAs discovered, there are discrepancies reported between the present and previous studies regarding the expression levels of miR-497-5p in breast cancer. Based on current observations, miR-497-5p was upregulated in the serum samples of breast cancer patients whereas several studies have reported the decreased expression of miR-497-5p in breast cancer tissue samples and cell lines. In a nude mouse xenograft tumour model, the inhibitory role of miR-497-5p in tumour growth and angiogenesis has been demonstrated while low miR-497-5p expression was associated with poor prognosis of breast cancer patients. For miR-377-3p, studies have shown that miR-377-3p was one of the miRNA transcripts that could predict tumour progesterone status with 100% accuracy and the Linc00339/miR-377-3p/HOXC6 axis represented a novel pathway in the progression of triple-negative breast cancer. MiR-374-5p has been shown to repress development of breast cancer through TATA-box binding protein associated factor 7 (TAF7)-mediated transcriptional regulation of DEP domain containing 1 (DEPDC1). The expression of miR-374-5p was downregulated in various breast cancer cell lines, similar to observation in this disclosure. A six-miRNA signature, which included miR-324-5p, had been shown to be associated with the reduced overall survival of triple-negative breast cancer. MiR-19b-3p has also been shown to be downregulated in hormone receptor-positive/HER2-negative breast cancer. With its high sensitivity and specificity in identifying breast cancer from healthy tissues and its involvement in regulation of genes in oncogenic pathways, miR-19b-3p can serve as a diagnostic marker or therapeutic target for breast cancer.

### Example 4: Sample Calculation of a Prediction Score

It is known in the art that, MiRNAs can be combined to form a biomarker panel to calculate the cancer risk score, for example using a linear model, for example, using a linear model. An example would be to calculate such a risk score using logistic regression, a form of linear model. The prediction score may also be calculated using a classification algorithm selected from the group comprising support vector machine algorithm, logistic regression algorithm, multinomial logistic regression algorithm, Fisher's linear discriminant algorithm, quadratic classifier algorithm, perceptron algorithm, k-nearest neighbours algorithm, artificial neural network algorithm, random forests algorithm, decision tree algorithm, naive Bayes algorithm, adaptive Bayes network algorithm, and ensemble learning method combining multiple 5 learning algorithms.

The challenge in the field pertains to identifying relevant biomarkers, such as circulatory miRNAs, that could be reliably applied to identify an individual at risk of a disease such as breast cancer. Where relevant miRNAs could be identified via exhaustive and well-designed studies, it would be within the skill of someone aware of the state of the art to apply the measured level of the relevant miRNAs in such statistical models to generate a score for the prediction of breast cancer. Formula 1 below exemplifies the use of a linear model for breast cancer risk prediction, where the cancer risk score (unique for each subject) indicates the likelihood of a subject having gastric cancer. This is calculated by the summing the weighted measurements for, for example, 8 miRNAs.

cancer risk score=C+Σᵢ₌₁¹²Kᵢ×log₂ copy_miRNA₁ Formula 1

log₂ copy_miRNAᵢ—log transformed copy numbers (copy/ml of serum/plasma) of the 8 individual miRNAs'). Whereby, Kᵢ—the coefficients used to weight multiple miRNA targets and C—constant, can be derived through the application of a linear model. The values of K, were optimized with support vector machine method and scaled to range from 0 to 100. Subjects with cancer risk score lower than 0 will be considered as 0 and subjects with cancer risk score higher than 100 will be considered as 100.

Examples of such mathematical methods used to perform the calculations disclosed herein, for example, the calculation of a prediction score, can be, but are not limited to, support vector machine algorithm, logistic regression algorithm, multinomial logistic regression algorithm, Fisher's linear discriminant algorithm, quadratic classifier algorithm, perceptron algorithm, k-nearest neighbours algorithm, artificial neural network algorithm, random forests algorithm, decision tree algorithm, naive Bayes algorithm, adaptive Bayes network algorithm, and ensemble learning method combining multiple learning algorithms. In one example, the calculation of the prediction score is calculated using linear models and support vector machine algorithms.

As an illustrative example, the control and cancer subjects in these studies have different cancer risk score values calculated based on the formula shown above. Fitted probability distributions of the cancer risk scores for the control and cancer subjects show a clear separation between the two groups can be found. Based on this prior probability and the fitted probability distributions previously determined, the probability (risk) of an unknown subject having cancer can be calculated based on their cancer risk score values. With higher score, the subject has higher risk of having breast cancer. Furthermore, the cancer risk score can, for example, tell the fold change of the probability (risk) of an unknown subject having breast cancer compared to, for example, the cancer incidence rate in high-risk population.

A requirement for the success of such process is the availability of high-quality data. The quantitative data of all the detected miRNAs in a large number of well-defined clinical samples not only improves the accuracy, as well as precision, of the result, but also ensures the consistency of the identified biomarker panels for further clinical application using quantitative polymerase chain reaction (qPCR).

### Example 5: Breast Cancer Prediction Algorithm Based on miRNA Biomarker Signature

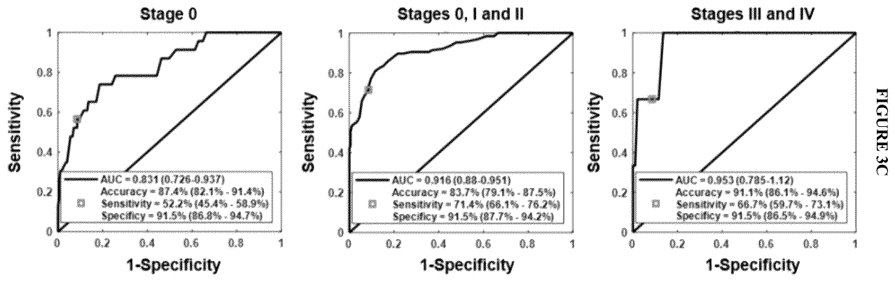

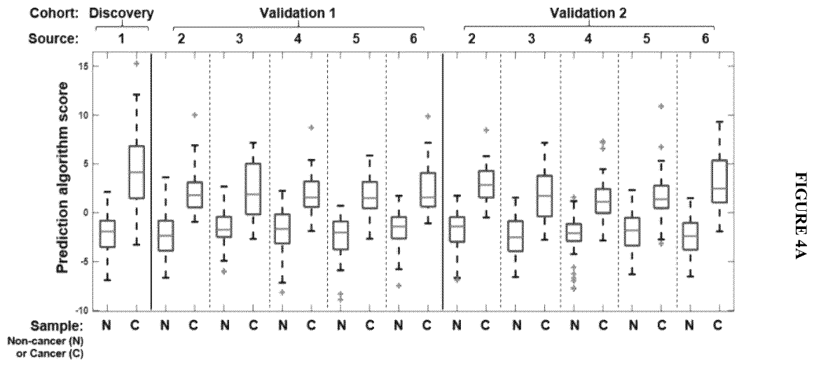

A prediction algorithm based on a logistic regression model that takes into account the expression levels of the eight miRNAs in the biomarker panel was developed to calculate a cancer risk score based on the expression of the eight miRNAs in the biomarker panel. Using this cancer risk score, cancer samples could be identified from non-cancer samples in all cohorts regardless of sample source (FIG. 4A). The panel effectively detects breast cancer of all stages, including early stage breast cancers (stages 0, I and II) (FIG. 3C), with cancer risk scores from breast cancer samples of all stages falling in the same range that is higher than that of non-cancer samples (FIG. 4B). The distribution of breast cancer samples by stage in each cohort is shown in Table 1.

### EXPERIMENTAL SECTION

### Blood Collection and Serum Processing

Peripheral blood samples (20 ml) were drawn from subjects using venipuncture and collected in serum tubes. Blood samples were clotted for 30 to 60 minutes and were centrifuged at 1,300 ref at room temperature for 20 minutes. Sera were then aliquoted for immediate storage at −80° C.

### RNA Isolation

Total RNA was extracted from 200 μl of each serum sample using the miRNeasy Serum/Plasma Kit (Qiagen, Hilden, Germany). This was done according to the manufacturer's recommendations, except for the following modifications: (a) a set of three proprietary spike-in controls (MiRXES, Singapore) was added, representing high, medium, and low levels of RNA, into the sample lysis buffer (QIAzol Lysis Reagent, Qiagen) prior to sample RNA isolation. The spike-in controls are 20-nucleotide RNAs with unique sequences (distinct from any of the 2588 annotated mature human miRNAs in miRBase version 21.0, RRID:SCR_003152) and are used to monitor RNA isolation efficiency and normalize for technical variations during RNA isolation; (b) bacteriophage MS2 RNA was added into sample lysis buffer (1 gg per ml of QiaZol) to improve RNA isolation yield; (c) the samples were centrifuged at 18,000×g for 15 minutes at room temperature after mixing with chloroform; and finally, (d) the RNA was eluted in 25 μl of RNase-free water.

RT-qPCR Detection of miRNA Expression

For biomarker discovery, a highly controlled RT-qPCR workflow was used to quantify the expression of 324 miRNAs in each serum sample. Serum RNA was reverse transcribed using miRNA-specific reverse transcription (RT) primers according to manufacturer's instructions (MiRXES) on a Veriti™ Thermal Cycler (Applied Biosystems, Foster City, CA, USA). Multiplexed RT reactions were carried out using specific RT primers for 324 miRNAs. This proprietary list of 324 circulating miRNAs was selected based on experimental analysis of more than 1000 high confidence human miRNAs from several hundred serum and plasma specimens. These 324 miRNAs are therefore those which have been detected with high confidence in human serum and plasma samples. The RT primers were divided into 10 multi-plex primer pools (50-60-plex per pool) to minimize non-specific crossovers and primer-primer interactions. For each RNA sample, 10 multiplex RT reactions were performed, each with 2 μl of isolated RNA. Synthetic templates for standard curves of each miRNA (6-log serial dilution of 10 million to 100 copies) and a non-template control (nuclease-free water spiked with MS2) were reverse transcribed concurrently with the isolated sample RNA. Synthetic miRNA standard curves were used to absolutely quantify sample miRNA expression copy numbers. To measure 324 miRNAs using quantitative PCR (qPCR), all cDNAs, including those from synthetic miRNA standards, were pre-amplified using a 14-cycle PCR reaction with Augmentation Primer Pools (MiRXES) on the Veriti™ Thermal Cycler. Single-plex qPCR was then performed on the amplified cDNA samples using a miRNA-specific qPCR assay (MiRXES) and ID3EAL miRNA qPCR Master Mix according to manufacturer's instructions (MiRXES). The qPCR reactions with technical duplicates were carried out on the ViiA™ qPCR system (384-well configuration, Applied Biosystems). Raw threshold cycle (Ct) values were calculated using the ViiA™ 7 RUO software with automatic baseline setting and a threshold of 0.5. RT-qPCR efficiency and potential cDNA amplification bias were assessed by analyzing the Ct values of the synthetic miRNA standards. The absolute expression of each miRNA (number of copies present) in the serum sample was calculated by intrapolation of sample Ct values with synthetic miRNA standard curves and correcting for variations in RT-qPCR efficiency. For biomarker validation, miRNA expression was quantified using the same workflow described above, adjusted for the number of miRNAs to be quantified.

### Biomarker Panel Building and Optimization

A two-fold cross-validation procedure that incorporated the sequential forward floating search (SFFS) algorithm and a logistic regression model was used for building and optimizing miRNA biomarker panels to discriminate between breast cancer cases and non-cancer controls. The SFFS was used to select miRNA biomarkers for inclusion in each biomarker panel built. In each iteration of the two-fold cross validation procedure, the samples included in the combined Discovery and Validation 1 cohorts (comprising a total of 663 samples from six sources) were randomly partitioned into two equal groups: Group A and Group B. The proportion of subjects from each of the six sources were partitioned equally in both Group A and B. During each iteration of cross-validation, Group A was first used as the training set for building a breast cancer prediction model while Group B was used as the test set. The group assignments as training and testing sets were then swapped. For every multi-miRNA biomarker panel optimized in each iteration, a logistic regression prediction model was built, and the diagnostic ability of each panel was evaluated using the area under the curve of the receiver operating characteristics (AUC) analysis. The cross-validation procedure was carried out 200 times. Thus, 200 two-miRNA panels, 200 three-miRNA panels, and so on, were optimized and tested. The diagnostic power (AUC) of each optimized multi-miRNA panel for classifying breast cancer and non-cancer patient samples was then calculated and compared with other panels optimized in each iteration. Using a logistic regression model incorporating multi-miRNA biomarker panel expression measurements, a prediction algorithm score could be calculated for each sample, with higher scores indicating increased risk of cancer. A prediction algorithm score cut-off was then used to predict breast cancer.

1. A method for determining whether a subject is suffering from, or is at risk of developing breast cancer, wherein the method comprises detecting differential expression levels of at least two or more miRNA markers from a biological sample obtained from the subject, wherein the miRNAs are selected from miR-133a-3p, miR-497-5p, miR-24-3p, miR-125b-5p, miR-377-3p, miR-374c-5p, miR-324-5p and miR-19b-3p, and wherein said differential expression level is compared with that of a cancer-free subject.

2. The method of claim 1, wherein the method comprises detecting at least 3, at least 4, at least 5, at least 6, at least 7, or at least 8 miRNA markers.

3. The method of any one of the preceding claims, wherein the at least 8 miRNA markers are selected from miR-133a-3p, miR-497-5p, miR-24-3p, miR-125b-5p, miR-377-3p, miR-374c-5p, miR-324-5p and miR-19b-3p.

4. The method of any one of the preceding claims, wherein the biological sample obtained from the subject is a bodily fluid.

5. The method of claim 4, wherein the bodily fluid is selected from the group consisting of cellular and/or non-cellular components of a liquid biopsy, amniotic fluid, a bronchial lavage, cerebrospinal fluid, interstitial fluid, peritoneal fluid, pleural fluid, saliva, seminal fluid, urine, a tear, peripheral blood, whole blood, plasma, and serum.

6. The method of claim 5, wherein the bodily fluid is serum.

7. The method of any one of the preceding claims, wherein the method is used in combination with one or more methods selected from the group consisting of mammography, ultrasound, resonance imaging or biopsy.

8. The method of any one of the preceding claims, wherein the differential expression is based on up- and/or downregulation of the miRNA, wherein, if present, the following miRNA are upregulated in a subject suffering from, or at risk of developing a breast cancer: miR-133a-3p, miR-497-5p, mir-24-3p, and miR-125b-5p.

9. The method of any one of the preceding claims, wherein the differential expression is based on up- and/or downregulation of the miRNA, wherein, if present, the following miRNA are downregulated in a subject suffering from, or at risk of developing breast cancer: miR-377-3p, miR-374c-5p, miR-324-5p and miR-19b-3p.

10. The method of any one of the preceding claims, wherein the subject is treated against breast cancer or the onset of breast cancer with anyone or more of the following anti-breast cancer treatments: surgery, radiation therapy, chemotherapy, hormone therapy, targeted therapy, immunotherapy or one or more anti-breast cancer compounds, when the subject is determined to have breast cancer or determined to be at a risk of developing breast cancer.

11. A method of treating breast cancer, wherein the method comprises:
i. detecting the presence of miRNA in a bodily fluid sample obtained from the subject;
ii. measuring the expression level of at least two miRNA in the bodily fluid sample; and
iii. using a prediction algorithm score based on the differential expression level of the miRNAs measured previously to predict the probability of the subject to suffer from or develop breast cancer,
wherein the at least two or more miRNA markers are selected from miR-133a-3p, miR-497-5p, miR-24-3p, miR-125b-5p, miR-377-3p, miR-374c-5p, miR-324-5p and miR-19b-3p,
and wherein the differential expression of miR-377-3p, miR-374c-5p, miR-324-5p and miR-19b-3p, if present, are downregulated, as compared to a control, or
wherein the differential expression of miR-133a-3p, miR-497-5p, mir-24-3p, and miR-125b-5p, if present, are upregulated, as compared to a control,
and determining the subject to suffer from breast cancer or to be at risk of developing breast cancer, and treating the subject determined to suffer from breast cancer or determined to be at risk of developing breast cancer with an anti-breast cancer compound,
wherein the control for comparing the expression level of the at least two miRNAs referred to in step ii) is a breast cancer-free subject.

12. The method of claim 11, wherein the prediction score is calculated using a classification algorithm selected from the group consisting of support vector machine algorithm, logistic regression algorithm, multinomial logistic regression algorithm, Fisher's linear discriminant algorithm, quadratic classifier algorithm, perceptron algorithm, k-nearest neighbours algorithm, artificial neural network algorithm, random forests algorithm, decision tree algorithm, naive Bayes algorithm, adaptive Bayes network algorithm, and ensemble learning method combining multiple learning algorithms.

13. A kit for use in the method according to any one of claims 1 to 12.

14. The kit of claim 13 for use in biochemical methods selected from the group consisting of a quantitative reverse-transcription real-time polymerase chain reaction (qRT-PCR), a locked nucleic acid (LNA) real-time PCR, sequencing, a northern blotting, a hybridization, a CRISPR gene editing, a micro-array assay, and combinations thereof.
